(19) United States
(12) Patent Application Publication
(10) Pub. No.: US 2022/0143143 A1
(43) Pub. Date: May 12, 2022

(54) USE OF ACTIVATORS OF THE ARYL HYDROCARBON RECEPTOR FOR TREATING GLUTEN-INDUCED GASTROINTESTINAL DISEASES

(71) Applicants: McMaster University, Hamilton (CA); Wageningen Universiteit, Wageningen (NL); Institut National De Recherche Pour L'Agriculture, L'Alimentation et I'Environnement, Paris (FR)

(72) Inventors: Jeremy Mark Wells, Zeist (NL); Philippe Langella, Velizy-Villacoublay (FR); Harry Sokol, Paris (FR); Elena Verdu, Ancaster (CA); Bruno Lamas, Tournefeuille (FR)

(21) Appl. No.: 17/429,297

(22) PCT Filed: Feb. 7, 2020
(86) PCT No.: PCT/CA2020/050168

§ 371 Date: Aug. 6, 2021

Related U.S. Application Data
(60) Provisional application No. 62/803,071, filed on Feb. 8, 2019.

(51) Int. Cl. A61K 38/20; A61K 31/405; A61K 31/553; A61K 31/404; A61K 31/198; A61K 31/352; A61K 31/353; A61K 31/522; A61K 31/465; A61K 31/357; A61K 31/137; A61K 35/741; A61K 35/745; A61K 35/747; A61K 35/744; A61P 3/00

(52) U.S. Cl. CPC A61K 38/20; A61P 3/00; A61K 31/553; A61K 31/404; A61K 31/198; A61K 31/352; A61K 31/353; A61K 31/522; A61K 31/465; A61K 31/357; A61K 31/137; A61K 35/741; A61K 35/745; A61K 35/747; A61K 35/744; A61K 31/405

(57) ABSTRACT

A method for treating a gluten-induced disease, such as celiac disease, in a subject in need thereof is provided. The method comprises administering to the subject at least one agent that activates aryl hydrocarbon receptor such as AhR agonists, bacterial probiotics with AhR agonist activity, and IL-22 agonists, polypeptides and nucleic acid.

### FIELD OF THE INVENTION

The present invention relates to methods for preventing or treating gastrointestinal diseases, and in particular, to methods of treating gluten-related conditions such as celiac disease.

### BACKGROUND

Gluten-related disorders are increasingly prevalent conditions that encompass all diseases triggered by dietary gluten, including celiac disease (CeD), a T-cell—mediated enteropathy, dermatitis herpetiformis, gluten ataxia, and other forms of non-autoimmune reactions. CeD is a chronic, autoimmune enteropathy caused by unknown environmental factors and triggered by gluten in individuals expressing HLA-DQ2 or DQ8. Up to 40% of most populations express the susceptibility genes for CeD; however, only 2%-4% will develop the disease, possibly due to additional unknown environmental triggers. Currently, a strict life-long gluten-free diet is the only efficient treatment available for CeD, which is financially and socially difficult for these patients.

Gluten proteins, predominantly gliadins in wheat, are resistant to complete degradation by mammalian enzymes, which results in the production of large peptides with immunogenic sequences. Partially digested gluten peptides translocate the mucosal barrier and are deamidated by human transglutaminase 2 (TG2), the CeD-associated autoantigen. This process converts glutamine residues to glutamate and increases peptide binding affinity to HLA-DQ2 or DQ8 heterodimers in antigen-presenting cells, initiating the T-cell mediated inflammation characteristic of CeD. NOD-DQ8 mice are mouse models that mimic aspects of the pathogenesis of CeD that develop moderate inflammation in the small intestine when sensitized and challenged with gluten.

There is little mechanistic insight behind the links between dysbiosis and gluten-specific T-cell responses, and the functional relevance of these associations in CeD are unclear. Innate and adaptive immune mechanisms are involved in the pathogenesis of CeD but the triggers or modulators of the innate immune pathway and cytotoxic intraepithelial lymphocytes (IEL) transformation remain unclear.

Aryl hydrocarbon receptors (AhR) are ligand-activated nuclear transcription factors and for many years, AhRs were exclusively studied for their role in mediating the toxicity of xenobiotics. Recently, they have been described as a player in immune response at barrier sites such as skin and mucosa. The makeup of the microbial community in the human gastrointestinal tract and dysbiosis, and their effects on AhR activity and signaling has also been studied with respect to the pathology of inflammatory bowel disease (IBD).

It would be desirable to gain further insight into the pathogenesis of gluten-related disorders and to develop methods of treatment for gluten-related disorders such as CeD.

### SUMMARY

The present application describes the identification of new mechanisms involved in the pathogenesis of gluten-induced disease. In particular, it has been determined that aryl hydrocarbon receptors play a role. It is herein demonstrated that the aryl hydrocarbon receptor (AhR) signaling pathway is disrupted in patients with a gluten-induced disease, and that an AhR agonist and compounds with like function can ameliorate gluten-induced disease.

Accordingly, in one aspect, a method for treating a gluten-induced disease in a subject in need thereof is provided comprising administering to the subject at least one agent that activates aryl hydrocarbon receptor.

In another aspect of the invention, a composition useful to treat a gluten-induced disease is provided comprising an AhR agonist and a bacterial probiotic that activates AhR.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF THE FIGURES

Embodiments of the invention are described in greater detail with reference to the attached figures in which:

FIG. 1 shows: A) Protocol for testing the effects of tryptophan (Trp) supplementation in NOD/DQ8 mice; B) Principal coordinate analysis based on bacterial 16S rRNA gene sequence abundance in fecal content of NOD/DQ8 mice fed with low (n=10) or enriched tryptophan diet (n=9) based on UniFrac distance matrices; and C) Alpha-diversity of fecal microbiota of NOD/DQ8 mice fed a low (n=10) or enriched tryptophan (Trp) diet (n=9).

FIG. 2 shows: A) Heat map of the significant fecal species between low (n=10) and enriched tryptophan diets (n=10) in NOD/DQ8 mice based on the total relative abundance (Unc.=unclassified bacteria); B) Relative abundance, at the phylum level, in NOD/DQ8 mice fed with low (n=10) or enriched tryptophan diet (n=9); and C) Diet-specific fecal changes in Lactobacillus relative abundance.

FIG. 3 shows: A) Quantification of tryptophan in feces of NOD/DQ8 mice fed with low (n=7) or enriched tryptophan diet (n=7); as well as fecal quantification of the AhR agonists: (B) tryptamine, (C) indole-3-aldehyde, and (D) indole-3-lactic acid, in NOD/DQ8 mice fed a low (n=6-7) or enriched tryptophan diet (n=6-7).

FIG. 4 shows serum quantification of: (A) tryptophan, (B) tryptamine, (C) indole-3-aldehyde and (D) indole-3-lactic acid) in NOD/DQ8 mice fed a low (n=6-7) or enriched tryptophan diet (n=6-7); and E) Quantification of kynurenine and IDO activity (calculated by kynurenine/tryptophan ratio) in feces of NOD/DQ8 mice fed with low (n=7) or enriched tryptophan diet (n=6-7).

FIG. 5 shows: A) AhR activity in feces and small intestine (SI) content of NOD/DQ8 mice fed with low (n=7-10) or enriched tryptophan diet (n=7-8); and B) Gene expression of the AhR pathway in the proximal small intestine of NOD/DQ8 mice fed with low (n=6-7) or enriched tryptophan diet (n=5-7).

FIG. 6 shows: A) Fecal lipocalin-2 quantification of NOD/DQ8 mice fed with low (n=8) or enriched tryptophan diet (n=7; B) Small intestinal CD3⁺ intraepithelial lymphocytes (IEL) counts (IEL/100 enterocytes) in NOD/DQ8 mice fed with low (n=6) or enriched tryptophan diet (n=5); and C-D) Small intestinal barrier function assessed by (C) ion secretion (μA/cm²) and (D) paracellular permeability to ⁵¹Cr-EDTA (% hot sample/h/cm²) in NOD/DQ8 mice fed with low (n=6-8) or enriched tryptophan diet (n=6-7).

FIG. 7 shows: A) Gluten sensitization and diet protocol for testing tryptophan (Trp) supplementation in gluten treated NOD/DQ8 mice; B) Principal coordinate analysis based on bacterial 16S rRNA gene sequence abundance in fecal content of NOD/DQ8 mice fed with low or enriched tryptophan (Trp) diet before (D21), and after gluten treatment (D59), based on UniFrac distance matrices; C) Alpha-diversity of fecal microbiota of NOD/DQ8 mice fed with low tryptophan diet before (D21; n=10) and after gluten treatment (D59; n=10); and, D) Alpha-diversity of fecal microbiota of NOD/DQ8 mice fed enriched tryptophan diet before (D21; n=9) and after gluten treatment (D59; n=8).

FIG. 8 shows: A) Relative abundance, at the phylum level, in NOD/DQ8 mice fed a low tryptophan diet before (D21; n=10), and after gluten treatment (D59; n=10); B) Relative abundance, at the phylum level, in NOD/DQ8 mice fed enriched tryptophan diet before (D21; n=9), and after gluten treatment (D59; n=8); C) Heat map of the significant species in feces of NOD/DQ8 mice fed a low tryptophan diet before (D21) and after gluten treatment (D59); and D) Heat map of the significant species in feces of NOD/DQ8 mice fed enriched tryptophan diet before (D21), and after gluten treatment (D59).

FIG. 9 shows: A) Alpha-diversity of fecal microbiota of gluten treated NOD/DQ8 mice fed a low (n=10) or enriched tryptophan (Trp) diet (n=8); B) Principal coordinate analysis based on bacterial 16S rRNA gene sequence abundance in fecal content of gluten treated NOD/DQ8 mice fed a low (n=10) or enriched tryptophan diet (n=8) using UniFrac distance matrices; and C) Heat map of the significant fecal species between gluten treated NOD/DQ8 mice fed low (n=10) or enriched tryptophan diet (n=8), based on the total relative abundance; and D) Relative abundance, at the phylum level, in gluten treated NOD/DQ8 mice fed with low (n=8) or enriched tryptophan diet (n=8).

FIG. 10 shows: A) Quantification of tryptophan and total AhR agonists (tryptamine, indole-3-aldehyde and indole-3-lactic acid) in feces of gluten treated NOD/DQ8 mice fed a low (n=9) or enriched tryptophan diet (n=8). B-C) Serum quantification of (B) tryptophan and (C) total AhR agonists (tryptamine, indole-3-aldehyde and indole-3-lactic acid) in gluten treated NOD/DQ8 mice fed a low (n=10) or enriched tryptophan diet (n=8); and D) Quantification of kynurenine and IDO activity, calculated by kynurenine/tryptophan ratio, in feces of gluten treated NOD/DQ8 mice fed a low (n=8-9) or enriched tryptophan diet (n=8).

FIG. 11 shows: A) AhR activity in feces and small intestine (SI) content of gluten treated NOD/DQ8 mice fed a low (n=8-9) or enriched tryptophan diet (n=8); and B) Gene expression of the AhR pathway in the proximal small intestine of gluten treated NOD/DQ8 mice fed a low (n=5-9) or enriched tryptophan diet (n=6-8).

FIG. 12 shows: A) Villus-to-crypt ratios in gluten treated NOD/DQ8 mice fed a low (n=8) or enriched tryptophan diet (n=7); B) Small intestinal CD3⁺ IEL counts (IEL/100 enterocytes) in gluten treated NOD/DQ8 mice fed a low (n=6) or enriched tryptophan diet (n=6); C) Small intestinal paracellular permeability to ⁵¹Cr-EDTA (% hot sample/h/cm²) in gluten treated NOD/DQ8 mice fed with low (n=11) or enriched tryptophan diet (n=12); D) Fecal lipocalin-2 quantification in gluten treated NOD/DQ8 mice fed a low (n=14) or enriched tryptophan diet (n=14); and E) Small intestinal barrier function assessed by ion secretion (μA/cm2) in gluten treated NOD/DQ8 mice fed a low (n=11) or enriched tryptophan diet (n=12).

FIG. 13 shows: A) Quantification of total AhR agonists (tryptamine, indole-3-aldehyde and indole-3-lactic acid) in fecal samples in patients with active celiac disease (CeD; n=10) and non-celiac controls (n=13). B-D) Quantification of (B) tryptamine (C) indole-3-aldehyde and (D) indole-3-lactic acid in fecal samples of patients with active celiac disease (CeD; n=9-10) and non-celiac controls (n=13).

FIG. 14: shows A-B) Quantification of (A) tryptophan and (B) total kynurenine metabolites (xanthurenic and kynurenic acid) in fecal samples in patients with active celiac disease (CeD; n=10) and non-celiac controls (n=13). C) IDO activity calculated by kynurenine/tryptophan ratio in fecal samples in patients with CeD (n=10) and non-celiac controls (n=13). D) Quantification of xanthurenic acid and (F) kynurenic acid in fecal samples of patients with active celiac disease (CeD; n=9-10) and non-celiac controls (n=13). E) AhR activity in fecal samples of patients with CeD (n=11) and non-celiac controls (n=14).

FIG. 15 shows gene expression of the AhR pathway in duodenal biopsy from patients with CeD (n=5) and non-celiac controls (n=5).

FIG. 16 shows: A) Protocol for testing a pharmacological AhR agonist (6-formylindolo [3, 2-b] carbazole, Ficz) in gluten treated NOD-DQ8 mice; B) Small intestine CD3⁺ IEL counts (IEL/100 enterocytes) in gluten treated NOD/DQ8 mice receiving a pharmacological AhR agonist (n=8) or vehicle (Day 40; n=8) and compared with non-sensitized mice at day 0 (n=8); C) Villus-to-crypt ratios in gluten treated NOD/DQ8 mice receiving a pharmacological AhR agonist (n=6) or vehicle (n=6) and compared with non-sensitized mice at day 0 (n=6); D) Gene expression of the AhR pathway in the proximal small intestine of gluten treated NOD/DQ8 mice receiving a pharmacological AhR agonist (n=7-8) or vehicle (n=7-8) and compared with non-sensitized mice at day 0 (n=6-8); and E-F) Small intestinal barrier function assessed by (E) paracellular permeability to ⁵¹Cr-EDTA flux (% hot sample/h/cm²) and (F) ion secretion (μA/cm²) in gluten treated NOD/DQ8 mice receiving a pharmacological AhR agonist (n=7) or vehicle (n=8) and compared with non-sensitized mice at day 0 (n=7-8).

FIG. 17 shows quantification of fecal lipocalin-2 in gluten treated NOD/DQ8 mice receiving a pharmacological AhR agonist (n=5) or vehicle (n=5).

FIG. 18 shows AhR activation by culture supernatants (2%, 10% and 20%) from L. reuteri F6 and L. reuteri A6 relative to that by supernatants from L. helveticus CNRZ 450, a lactobacillus strain which does not produce AhR ligands.

FIG. 19 shows: A) Protocol for dietary tryptophan (Trp) and L. reuteri effects. B) AhR activity in the small intestinal (SI) content of gluten treated NOD/DQ8 mice with and without L. reuteri and fed a low (n=4-5/group) or enriched tryptophan diet (n=4-5/group). C) Small intestinal CD3⁺ IEL counts (IEL/100 enterocytes) in gluten treated NOD/DQ8 mice with and without L. reuteri and fed a low (n=5/group) or enriched tryptophan diet (n=5/group). D) Villus-to-crypt ratios in gluten treated NOD/DQ8 mice with and without L. reuteri and fed a low (n=5/group) or enriched tryptophan diet (n=5-6/group).

FIG. 20 illustrates: A) the protein sequence of human AhR, and B) the transcript sequence of human AhR.

FIG. 21 illustrates: A) the protein sequence of human IL-22, and B) the transcript sequence of human IL-22.

### DETAILED DESCRIPTION

In one aspect, a method for treating a gluten-induced disease in a subject in need thereof is provided comprising administering to the subject at least one agent that activates aryl hydrocarbon receptor.

As used herein, the term “gluten-induced disease” has its general meaning in the art and refers to a group of gluten-induced diseases and disorders such as celiac disease (CeD), a T-cell˜mediated enteropathy, dermatitis herpetiformis, gluten ataxia, non-celiac gluten or wheat sensitivity and other non-autoimmune reactions.

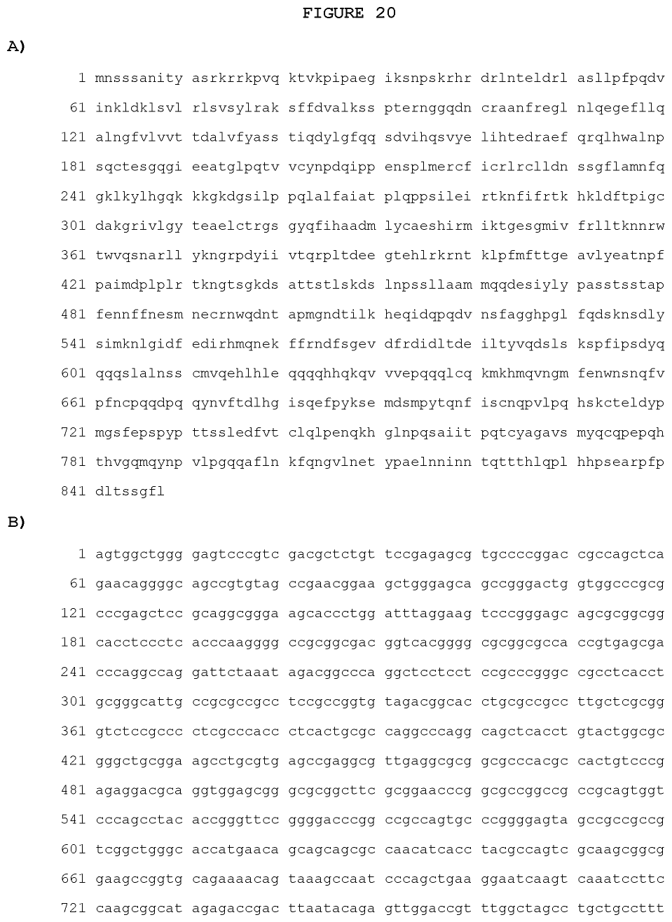

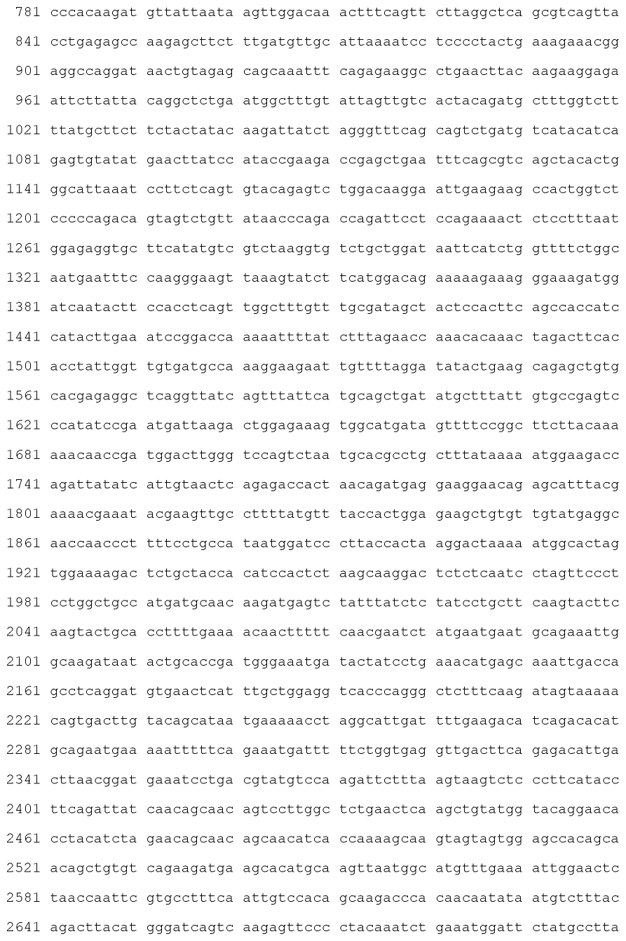

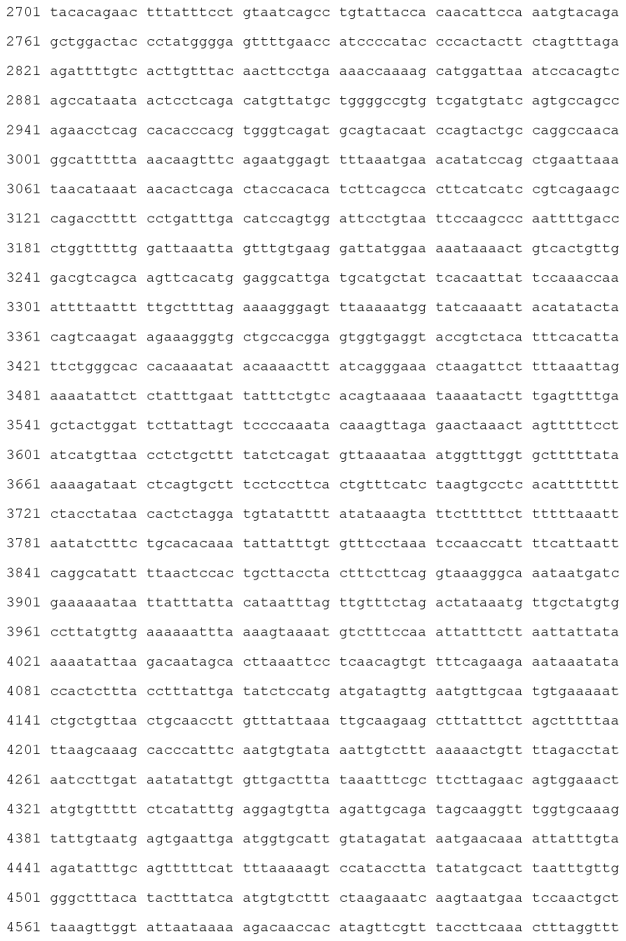

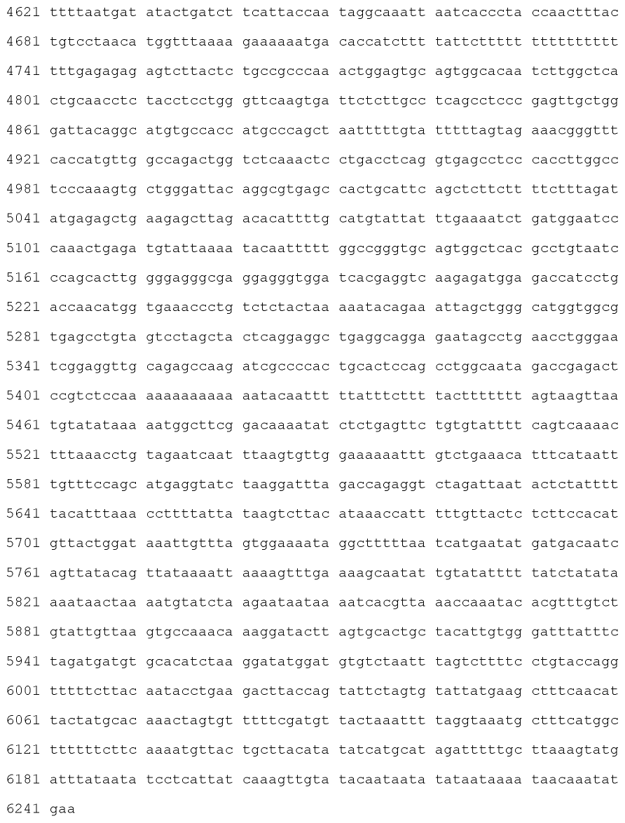

The term “aryl hydrocarbon receptor” or “AhR” refers to a transcription factor which is activated by a diverse range of compounds and regulates the expression of xenobiotic metabolism genes. Aryl hydrocarbon receptor (AhR) is a member of the family of basic helix-loop-helix transcription factors, the bHLH-PAS (basic helix-loop-helix/Per-ARNT-Sim) family. As used herein, AhR encompasses mammalian AhR, including human and non-human AhR. Human AhR is depicted by the protein sequence shown in FIG. 20A (NCBI Reference Sequence: NP_001612.1) encoded by the transcript shown in FIG. 20B (NCBI Reference Sequence: NM_001621.5). Non-human sequences are also known as depicted, for example, by NCBI Reference Sequence NP_001300956 and NP_038492. As one of skill in the art will appreciate, functionally equivalent variant forms of this protein may exist, comprising amino acid insertions, deletions or substitutions, such as conservative amino acid substitutions, which retain AhR activity.

The term “AhR activity” has its general meaning in the art and refers to the biological activity associated with the activation of the AhR resulting from its signal transduction cascade, including any of the downstream biological effects resulting from the binding of a natural AhR ligand (e.g. endogenous AhR activity), or an agonist or candidate agent in accordance with the embodiments described herein. As one of skill in the art will appreciate, AhR activity resulting from the binding of an agonist or other agent may vary from the activity resulting from the binding of the AhR to a natural ligand thereof, for example, the AhR activity may be equal to, or higher or lower than the biological effect resulting from the binding of the AhR to one or more of its natural ligands. Preferably, the AhR activity resulting from the binding of an agonist or other agent in accordance with embodiments of the invention will be at least about 20% of endogenous AhR activity, e.g. at least 30-50% of endogenous AhR activity or greater, including about 100% of endogenous AhR activity or more.

The agent that activates AhR may binds to the AhR to causes dissociation of the AhR from chaperone molecules to permit dimerization of the AhR with AhR nuclear translocator (ARNT) to increase AhR activity. An agent that binds to the AhR to result in dissociation of the AhR from chaperones and dimerization with ARNT is selected from the group comprising naturally occurring or synthetic AhR agonists. Other agents that activate AhR include bacterial probiotics with AhR agonist activity, IL-22, IL-22 agonists and IL-17 antagonists.

The term “AhR agonist” has its general meaning in the art and refers to a compound or agent that activates the AhR, preferably selectively activates the AhR. The term “AhR agonist” refers to natural AhR ligands and any compound or agent that can directly or indirectly stimulate the signal transduction cascade related to the AhR. As used herein, the term “selectively activates” refers to a compound or agent that preferentially binds to and activates AhR with a greater affinity and potency, respectively, than its interaction with the other members of bHLH-PAS transcription factors family. Compounds or agents that prefer AhR, but that may also activate other sub-types, as partial or full agonists are also contemplated. Typically, an AhR agonist is a small organic molecule or a peptide.

In one embodiment of the invention, the agent is an AhR agonist and may be a naturally occurring or synthetic molecule or a mixture, such as a botanical extract, that directly interacts with the AhR protein, inducing its dissociation from chaperone proteins (e.g. consisting of a dimer of Hsp90 and prostaglandin E synthase 3 (PTGES3, p23), and a single molecule of the immunophilin-like AH receptor-interacting protein (e.g. hepatitis B virus X-associated protein 2 (XAP2), AhR interacting protein (AIP) or AhR-activated 9 (ARA9)). Dissociation of the AhR from the chaperone proteins results in its translocation into the nucleus and dimerization with ARNT (AhR nuclear translocator), leading to changes in target gene transcription (e.g. such as transcription of cytochrome P450, family 1, subfamily A, polypeptide 1 (Cyp1a1), and Phase I and Phase II metabolizing enzymes consisting of CYP1A1, CYP1A2, CYP1B1, NQO1, ALDH3A1, UGT1A2 and GSTA1) to produce a physiological effect.

Examples of agonists of AhR include, but are not limited to, indole derivatives such as indolocarbazole (ICZ) and 6-formylindolo(3,2-b)carbazole (Ficz), tryptophan and derivatives thereof, tryptophan catabolites such as tryptophan catabolites of the microbiota, e.g. kynurenine, kynurenic acid, indole-3-aldehyde (IAld), tryptamine, indole 3-acetate and 3-indoxyl sulfate, 2,3,7,8-tetrachlorodibenzo-p-dioxin (TCDD), flavonoids such as quercetin, diosmin, tangeritin, tamarixetin, luteolin and myricetin, polycyclic aromatic hydrocarbons, e.g. 3-methylcholanthrene, benzo[a]pyrene, benzanthracenes and benzoflavones, biphenyls and polyphenolics, and halogenated aromatic hydrocarbons (polychlorinated dibenzodioxins, dibenzofurans and biphenyls). Other AhR activators include caffeine, nicotine, pyridines, benzimadazoles and methylenedioxybenzenes. Natural AhR agonists (NAhRAs) include, but are not limited to, tryptophan derivatives such as indigo dye and indirubin, tetrapyrroles such as bilirubin, the arachidonic acid metabolites, lipoxin A4 and prostaglandin G modified low-density lipoprotein and several dietary carotenoids.

The AhR agonist may be a selective AhR modulator (SAhRM) such as diindolylmethane (DIM) and carbidopa, methyl-substituted diindolylmethanes, dihalo- and dialkylDIM analogs, mexiletine, β-naphthoflavone (PNF), 5,6 benzoflavone (5,6 BZF), 1,4-dihydroxy-2-naphthoic acid (DHNA), and moieties described, for example, in Safe et al, 2013; Furumatsu et al, 2011; and WO 2012/015914. The AhR agonist may additionally include compounds described in WO 2012/015914 such as CB7950998.

The AhR agonist also includes natural extracts or fractions which are activators of the AhR pathway such as 1,4-dihydroxy-2-naphthoic acid (DHNA) and natural AhR agonists (NAhRAs) disclosed in WO 2013/171696 and WO 2009/093207.

In another embodiment, the agent is a bacterial probiotic that itself activates AhR, or that produces an AhR-activating substance. The term “bacterial probiotic” has its general meaning in the art and refers to a useful microorganism or mixture of microorganisms that improve the bacterial flora in the gastrointestinal tract and can result in a beneficial action to the host, e.g. production of a growth-promoting substance. The term “bacterial probiotic” also refers to a bacterium forming the bacterial flora and may include a substance that promotes the growth of such a bacterium. The term “bacterial probiotic” also refers to a useful microorganism that can bring a beneficial action to a host, such as produce a substance that brings about desirable effect. A growth-promoting substance having AhR-activating potency includes a case in which the substance itself has AhR-activating potency and also a case in which the substance itself does not have AhR-activating potency but it promotes growth of a bacterium having AhR-activating potency. The term “bacterial probiotic” also refers to a dead microbial body and a microbial secretory substance. Because of a suitable enteric environment being formed and the action being independent of differences in enteric environment between individuals, the probiotic is preferably a living microbe. The term “bacterial probiotic exhibiting AhR activation properties” has its general meaning in the art and relates to a probiotic which can activate the AhR. The term “bacterial probiotic exhibiting AhR activation properties” also relates to a probiotic capable of activating the AhR or having AhR activating potency. The term “AhR activation properties” means potency in being able to activate a signaling pathway that is initiated by AhR activation, and may involve any kind of activating mechanism. Therefore, it is not always necessary for a microbial body itself to be an AhR ligand, but it may produce a secretory substance having AhR-activating potency, or the AhR may be activated by a dead microbial body or homogenate thereof. Therefore, when a “microorganism” or “bacterium” is referred to herein, or a specific microbe is referred to herein, this encompasses a living microbe and/or a dead microbial body or homogenate thereof, and/or a culture of said microbe and/or a secretory substance of said microbe. Preferably, a bacterial probiotic comprises a microbial body itself such as a living microbe or a dead microbial body or homogenate thereof, and more preferably the probiotic is a living microbe capable of forming bacterial flora in the gastrointestinal tract.

Bacterial probiotics with AhR agonist activity include, but are not limited to, bacterium naturally exhibiting AhR activation properties or modified bacterium exhibiting AhR activation properties such as, but not limited to, Allobaculum, Lactobacilli that produce AhR agonists, Lactobacilli such as Lactobacillus reuteri, Lactobacillus taiwanensis, Lactobacillus johnsonii, Lactobacillus animalis, Lactobacillus murinus, Lactobacillus bulgaricus, Lactobacillus delbrueckii subsp. bulgaricus, bacteria of the genus Adlercreutzia, bacteria of the phylum Actinobacteria, lactic acid bacterium, Streptococcus thermophilus, Bifidobacterium, Propionic acid bacterium, Bacteroides, Eubacterium, anaerobic Streptococcus, Enterococcus, Escherichia coli, other intestinal microorganisms and probiotics as described, for example, in US 2013/0302844, and combinations thereof.

In particular, five bacterial probiotics deposited at the Collection Nationale de Cultures de Microorganismes (CNCM, Institut Pasteur, 25 rue du Docteur Roux, 75724 Paris Cedex 15, France), in accordance with the terms of Budapest Treaty, on the 30 Sep. 2015, are useful as AhR agonists in accordance with the invention. The deposited bacterial probiotics have CNCM deposit numbers CNCM I-5019 (SB6WTD3, Lactobacillus taiwanensis), CNCM 1-5020 (SB6WTD4, Lactobacillus murinus), CNCM 1-5021 (SB6WTD5, Lactobacillus animalis), CNCM 1-5022 (SB6WTF6, Lactobacillus reuteri), and CNCM 1-5023 (SB6WTG6, Lactobacillus reuteri). Accordingly, the present invention also relates to a bacterial probiotic exhibiting AhR activation properties selected from the group consisting of bacterial probiotics available under CNCM deposit numbers CNCM 1-5019, CNCM 1-5020, CNCM 1-5021, CNCM 1-5022, CNCM 1-5023. Reference to these strains are described in WO 2017/032739.

Probiotics are advantageously used in conjunction with prebiotics to support the growth of the probiotic. Prebiotics may also be used in accordance with the present invention alone to stimulate the growth of gut bacteria having AhR-activating activity. Prebiotics generally comprise sources of fiber, e.g. undigestable plant fibers, particularly indole-rich foods. Thus, prebiotics may include foods such as apple, asparagus, onion, garlic, artichoke, leek, cabbage, broccoli, banana, grapefruit, chickory root, lentils, chick peas, other legumes, nuts and seeds such as almonds and flaxseed, and cereals (preferably non-gluten-containing).

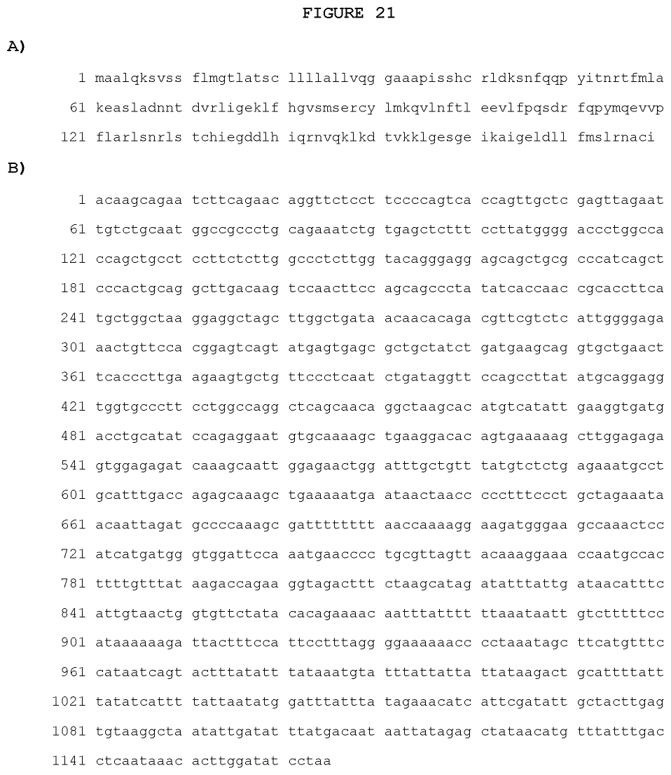

In one embodiment, the agent of the present invention is an IL-22 agonist, IL-22 polypeptide or nucleic acid encoding IL-22, which is useful to increase the level of IL-22 activity or expression. The term “IL-22 agonist” has its general meaning in the art and refers to compounds which enhance IL-22 expression, such as IL-22-Fc. The term “IL-22 polypeptide” has its general meaning in the art and includes naturally or non-naturally occurring IL-22 and functionally equivalent variants thereof which comprise amino acid insertions, deletions or substitutions (e.g. conservative amino acid substitutions) but which essentially retain the activity of IL-22. The IL-22, and nucleic acid encoding IL-22, can be from any source, but typically is a mammalian (e.g., human and non-human primate) IL-22, and more particularly a human IL-22. The amino acid sequence of human IL-22 is provided in FIG. 21A (NCBI Reference Sequence: NP_065386.1), while the transcript sequence is provided in FIG. 21B (NCBI Reference Sequence: NM_020525.5). Other mammalian IL-22 sequences are known, for example, NCBI Reference Sequence: NP_473420, and transcript sequences, e.g. NCBI Reference Sequence: NM_054079.

With respect to functionally equivalent variants of IL-22, these may include analogues, derivatives or fragments IL-22. Analogues of interleukin-22 may incorporate one or more amino acid substitutions, additions or deletions. Amino acid additions or deletions include both terminal and internal additions or deletions to yield a functionally equivalent peptide. Examples of suitable amino acid additions or deletions include those incurred at positions within the protein that are not closely linked to activity. Amino acid substitutions, particularly conservative amino acid substitutions, may also generate functionally equivalent analogues of IL-22. Examples of conservative substitutions include the substitution of a non-polar (hydrophobic) residue such as alanine, isoleucine, valine, leucine or methionine with another non-polar (hydrophobic) residue; the substitution of a polar (hydrophilic) residue with another such as between arginine and lysine, between glutamine and asparagine, between glutamine and glutamic acid, between asparagine and aspartic acid, and between glycine and serine; the substitution of a basic residue such as lysine, arginine or histidine with another basic residue; or the substitution of an acidic residue, such as aspartic acid or glutamic acid with another acidic residue. Functionally equivalent fragments of IL-22 comprise a portion of the IL-22 sequence which maintains the function of intact polypeptide, e.g. with respect to inducing AhR activity. Such biologically active fragments of interleukin-15 can readily be identified using assays as described herein. Functionally equivalent derivatives of interleukin-22 include IL-22, or an analogue or fragment thereof, in which one or more of the amino acid residues therein is chemically derivatized. The amino acids may be derivatized at the amino or carboxy groups, or alternatively, at the side “R” groups thereof. Derivatization of amino acids within the peptide may render a peptide having more desirable characteristics such as increased stability or activity. Such derivatized molecules include for example, those molecules in which free amino groups have been derivatized to form, for example, amine hydrochlorides, p-toluene sulfonyl groups, carbobenzoxy groups, t-butyloxycarbonyl groups, chloroacetyl groups or formyl groups. Free carboxyl groups may be derivatized to form, for example, salts, methyl and ethyl esters or other types of esters or hydrazides. Free hydroxyl groups may be derivatized to form, for example, O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids, for example: 4-hydroxyproline may be substituted for proline; 5-hydroxylysine may be substituted for lysine; 3-methylhistidine may be substituted for histidine; homoserine may be substituted for serine; and ornithine may be substituted for lysine. Terminal derivatization of the protein to protect against chemical or enzymatic degradation is also encompassed including acetylation at the N-terminus and amidation at the C-terminus of the peptide.

Interleukin-22, and functionally equivalent variants thereof, may be made using standard, well-established solid-phase peptide synthesis methods (SPPS), or techniques based on recombinant technology. It will be appreciated that such techniques are well-established by those skilled in the art, and involve the expression of interleukin-22-encoding nucleic acid in a genetically engineered host cell. DNA encoding IL-22 may be synthesized de novo by automated techniques well-known in the art given that the protein and nucleic acid sequences are known.

Interleukin-22-encoding nucleic acid molecules or oligonucleotides, and functionally equivalent forms thereof (e.g. that encode functionally equivalent interleukin-15, or nucleic acids which differ due to degeneracy of the genetic code) may also be used in the present methods. Encompassed are oligomers or polymers of nucleotide or nucleoside monomers consisting of naturally occurring bases, sugars, and intersugar (backbone) linkages. The term also includes modified or substituted oligonucleotides comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells), or two or more oligonucleotides of the invention may be joined to form a chimeric oligonucleotide. Other oligonucleotides of the invention may contain modified phosphorous, oxygen heteroatoms in the phosphate backbone, short chain alkyl or cycloalkyl intersugar linages or short chain heteroatomic or heterocyclic intersugar linkages. For example, oligonucleotides may contain phosphorothioates, phosphotriesters, methyl phosphonates, and phophorodithioates. Oligonucleotides of the invention may also comprise nucleotide analogs such as peptide nucleic acid (PNA) in which the deoxribose (or ribose) phosphate backbone in the DNA (or RNA), is replaced with a polymide backbone similar to that found in peptides. Other oligonucleotide analogues may contain nucleotides containing polymer backbones, cyclic backbones, or acyclic backbones, e.g. morpholino backbone structures.

Such oligonucleotide molecules are readily synthesized using procedures known in the art based on the available sequence information. For example, oligonucleotides may be chemically synthesized using naturally occurring nucleotides or modified nucleotides as described above designed to increase the biological stability of the molecules or to increase the physical stability of the duplex formed with mRNA or the native gene, e.g. phosphorothioate derivatives and acridine substituted nucleotides. Selected oligonucleotides may also be produced biologically using recombinant technology in which an expression vector, e.g. plasmid, phagemid or attenuated virus, is introduced into cells in which the oligonucleotide is produced under the control of a regulatory region.

IL-22 agonists are well-known in the art as illustrated by WO 2011087986 and WO 2014145016. IL-22 polypeptides are well-known in the art as illustrated by WO 2014/053481 and WO 2014/145016.

In one embodiment, the agent of the present invention is an IL-17 antagonist, a compound that inhibits the activity or expression of IL-17. Examples of IL-17 antagonists include, but are not limited to, ixekizumab, secukinumab and anti-IL-17-receptor antibodies such as brodalumab. Additional IL-17 antagonists that may be useful are well-known in the art as illustrated by WO 2013/186236, WO 2014/001368, WO 2012/059598, WO 2013/158821 and WO 2012/045848.

As one of skill in the art will appreciate, tests and assays for determining whether a compound is an AhR agonist are well known in the art such as described in Ji et al., 2015; Furumatsu et al., 2011; Gao et al, 2009; and WO 2012/015914). For example, in vitro and in vivo assays may be used to assess the potency and selectivity of a candidate agent to induce AhR activity. The activity of the candidate agent, its ability to bind AhR and its ability to induce similar effects to that of the indole derivatives, indole-3-aldehyde (IAld) or 6-formylindolo(3,2-b)carbazole (Ficz), may be tested using isolated cells expressing AhR, AhR-responsive recombinant cells, colonic and small intestine lamina proporia cells expressing AhR, Thl7/Th22 cells, γδT cells, NKp46+ILC cells, group 3 innate lymphoid cells (ILC3s) expressing the AhR, CHO cell line cloned and transfected in a stable manner by the human AhR or other tissues expressing AhR.

For example, the activity of a candidate agent and its ability to bind to the AhR may be assessed by the determination of a Ki on the AhR cloned and transfected in a stable manner into a CHO cell line as well as measuring the expression of AhR target genes, measuring Trp, Kyn and indole derivative (IAA) concentrations, measuring Kyn/Trp, IAA/Trp and Kyn/IAA concentration ratios, measuring IL-17+ and IL-22+ cells, measuring AhR and chaperone protein heterodimerization, measuring AhR nuclear translocation, or measuring AhR binding to its dimerization partner (AhR nuclear translocator (ARNT)) in the present or absence of the candidate agent. The activity of the candidate agent in cells, e.g. intestine cells, and other tissues expressing a receptor other than AhR may be used to assess selectivity of the candidate agents.

Accordingly, in another aspect of the invention, a method of screening a candidate agent for use as a drug for the prevention or treatment of a gluten-induced disease such as CeD in a subject in need thereof is, thus, provided. The method comprises the steps of: exposing a cell or tissue that expresses an AhR to a candidate agent such as a small organic molecule, peptide, polypeptide, non-peptide compound, peptide mimetic, metabolically and/or conformationally stabilized peptide analogs, derivatives or pseudo-peptides and probiotics under conditions suitable to maintain the cell or tissue in a viable state; measuring the AhR activity of the cell or tissue in the presence of the candidate agent; and identifying a candidate agent as a potential drug if the agent induces AhR activity in the cell or tissue.

To determine whether or not a subject requires treatment, the AhR activation or activity level of an appropriate biological sample from the subject may be assessed by any of a wide variety of well-known methods (He et al., 2011; Gao et al., 2009). The biological sample may be colon-related sample, e.g. feces or colon tissue, as well as serum, plasma, or small intestinal brushes or aspirates obtained during upper gastroduodenal endoscopy. The term “subject” is used herein to refer to a mammalian subject, including both human and non-human mammals such as domestic animals (cats, dogs, rodents, and the like) as well as livestock (e.g. cattle, horses, goats, sheep, pigs, etc.). Typically, a subject according to the invention refers to any subject (preferably human) afflicted with or susceptible to being afflicted with a gluten-induced disease. In a particular embodiment, the term “subject” refers to a subject afflicted with with Celiac Disease.

In one embodiment, the AhR activation level of the microbiota in a feces sample obtained from the subject is assessed using cell-based assays as described in the specific examples herein (He et al., 2011 and Gao et al., 2009). The AhR activation level within the sample may be assessed by determining the luciferase activity of AhR-responsive recombinant cells in which the luciferase reporter gene is functionally linked to an AhR-responsive promoter. Thus, quantifying changes in luciferase expression in the reporter cells in the presence of a sample provides a sensitive surrogate measure of the level of AhR activity in the sample. Examples of such cells include, but are not limited to, AhR-responsive recombinant guinea pig (G16L1.1c8), rat (H4L1.1c4), mouse (H1L1.1c2) and human (HG2L6.1c3) cells. The AhR activation level may also be assessed by measuring the ability of the sample to stimulate AhR-dependent gene expression using recombinant mouse hepatoma (Hepalclc7) cell-based CALUX (H1L1. 1c2 and H1L6. Ic2) clonal cell lines that contain a stably integrated AhR-/dioxin-responsive element (DRE)-driven firefly luciferase plasmid (pGudLucl. 1 or pGudLuc6.1, respectively) and CAFLUX (H1G1.1c3) clonal cell lines (He et al., 2011).

In one embodiment, the AhR activation level of the microbiota in a feces sample obtained from the subject is assessed by measuring tryptophan metabolism within the sample. Accordingly, the AhR activation level may be assessed by measuring Tryptophan (Trp), kynurenine (Kyn) and indole derivative (such as indole-3-acetic acid (IAA)) or other tryptophan metabolite concentrations, or measuring Kyn/Trp, IAA/Trp and/or Kyn/IAA concentration ratios.

In one embodiment, the AhR activation level is assessed using colon samples obtained from the subject by analyzing the expression of an AhR target gene (such as IL-22 and/or IL-17), by measuring IL-17+ and/or IL-22+ cell number, measuring AhR and chaperone protein heterodimerization, measuring AhR nuclear translocation, or measuring AhR binding to its dimerization partner (AhR nuclear translocator (ARNT)).

Once a subject is determined to be in need of treatment for CeD or risk of CeD, a therapeutically effective amount of at least one agent that increases AhR activity, i.e. activates AhR to a desirable, healthy level, is administered to the subject. A subject with CeD or at risk of developing CeD may have an AhR activation level of below the 50^{th} percentile found in a normal healthy (non-CeD) population. The therapeutically effective amount of the selected AhR agonist will vary with the selected agonist, as well as criteria such as age, symptoms, body weight, etc. of the subject. The term “therapeutically effective amount” is an amount of the AhR agonist required to activate AhR to a desired level, e.g. an amount sufficient to increase AhR activity to a level within the range of a healthy population with no or minimal levels of inflammation, while not exceeding an amount which may cause significant adverse effects. The terms “treat”, “treatment” and the like are used here to refer to the treatment, prevention, amelioration, reduction of symptoms and the like with respect to a gluten-induced disease.

The selected AhR agonist may be administered alone or in combination with pharmaceutically acceptable carriers, excipients, adjuvants, and the like for use in treatments in accordance with embodiments of the invention. The expression “pharmaceutically acceptable” means acceptable for use in the pharmaceutical and veterinary arts, i.e. not being unacceptably toxic or otherwise unsuitable, e.g. does not produce an adverse, allergic or other untoward reaction when administered to a mammal, especially a human, as appropriate. A pharmaceutically acceptable carrier, excipient or adjuvant refers to a non-toxic solid, semi-solid or liquid filler, diluent, encapsulating material or formulation auxiliary of any type. Examples are those used conventionally with each particular class of drug, e.g. peptide- or probiotic-based drugs. Reference may be made to “Remington's: The Science and Practice of Pharmacy”, 21st Ed., Lippincott Williams & Wilkins, 2005, for guidance on drug formulations generally. The selection of adjuvant depends on the intended mode of administration of the composition. In one embodiment of the invention, the compounds are formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule or suspension are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. Sustained-release matrices, such as biodegradable polymers, may also be utilized in the present compositions. Creams, lotions and ointments may be prepared for topical application using an appropriate base such as a triglyceride base. Such creams, lotions and ointments may also contain a surface active agent. Aerosol formulations may also be prepared in which suitable propellant adjuvants are used. Other adjuvants may also be added to the composition regardless of how it is to be administered, for example, anti-microbial agents may be added to the composition to prevent microbial growth over prolonged storage periods.

In the present treatment or prevention method, the AhR agonist may be administered by any route suitable to result in the desired AhR activation. Examples of suitable administrable routes include, but are not limited to, oral, subcutaneous, intravenous, intraperitoneal, intranasal, enteral, topical, sublingual, intramuscular, intra-arterial, intramedullary, intrathecal, inhalation, ocular, transdermal, vaginal or rectal means. Depending on the route of administration, the protein or nucleic acid may be coated or encased in a protective material to prevent undesirable degradation thereof by enzymes, acids or by other conditions that may affect the therapeutic activity thereof.

For probiotic treatments, the therapeutically effective amount ingested per day of the probiotic, or orally ingested composition of the present invention, is not particularly limited and may be appropriately adjusted according to criteria such as age, symptoms and body weight of the subject, and intended application. For example, the amount ingested per day of the probiotic is typically 0.01-100×10¹¹ cells/body (e.g. 10⁹-10¹¹ cells), preferably 0.1-10×10¹¹ cells/body, and more preferably 0.3-5×10¹¹ cells/body. Furthermore, for example, the amount ingested per day as the probiotic is 0.01-100×10¹¹ cells/60 kg body weight, preferably 0.1-10×10¹¹ cells/60 kg body weight, and more preferably 0.3-5×10¹¹ cells/60 kg body weight.

In one embodiment, the present method comprises the use of an oral composition comprising the bacterial probiotic for prevention and/or treatment of gluten-induced diseases. Typically, such an oral composition is combined with adjuvants acceptable for oral ingestion, and formulated to form a composition selected from the group consisting of a beverage or drink composition, a food composition, a feedstuff composition and a pharmaceutical composition. Thus, probiotics may be combined with milk and milk products including yogurt, pudding and shakes, juices, smoothies, soy products, gluten-free cereals, and other gluten-free products. The oral probiotic will typically comprise a dry probiotic microbial content in the range of about 1 to 95 w/w %, for example, 1 to 75 w/w % or 5 to 50 w/w %.

The bacterial probiotic may also be administered as a transplant composition, for example, a fecal microbiota transplant composition comprising the bacterial probiotic. The term “fecal microbiota transplant composition” has its general meaning in the art and refers to any composition that can restore the fecal microbiota to a healthy level. In a particular embodiment, the fecal microbiota transplant composition is a fresh or frozen stool sample from a healthy subject not afflicted with CeD.

Metabolism of tryptophan by gut microbiota and the host cell produce metabolites in the gastrointestinal (GI) tract that can act as ligands and agonists for the AhR pathway. The interplay of tryptophan metabolism by gut microbiota and the host cell results in levels of different tryptophan metabolites that can directly impact AhR pathway signaling. Disruption in the metabolism of tryptophan may lead to reduced AhR activation and contribute to the pathology of CeD. In CeD patients, it was determined that tryptophan metabolism in the GI tract differed from that of healthy individuals. Tryptophan and metabolites produced by microbiota metabolism were lower in CeD patients, while levels of tryptophan metabolites produced by host cell metabolism were higher. Supplementation with high levels of tryptophan, either prior to gluten sensitization or subsequent to gluten sensitization, increases expression of genes from the AhR pathway and elevates AhR activation. Thus, treatment of a subject with tryptophan, probiotic able to catabolize tryptophan, or a combination thereof is useful to mitigate pathologies induced by gluten sensitization. In this regard, dosage of each will be in a range suitable to activate AhR activity. For tryptophan, a dosage in the range of about 1-10 g per day, preferably in the range of about 1-6 g/day, or an amount in the range of about 3-5 mg/kg per day, e.g. 4 mg/kg per day.

As one of skill in the art will appreciate, a subject having or at risk of a gluten-induced disease may be treated with a combination of agents that function to increase AhR activity. Thus, an AhR agonist may be combined with a suitable probiotic treatment that increases AhR activity, or may be combined with IL-22, IL-22 agonist and/or IL-17 antagonist. Likewise, a probiotic treatment that activates AhR, may be combined with IL-22, IL-22 agonist and/or IL-17 antagonist. For example, an AhR agonist such as an indole, tryptophan or tryptophan catabolites may be combined with an AhR-activating bacterial probiotic, such as one or more of the bacterial probiotics identified herein, e.g. bacteria of the Lactobacillus genus, in accordance with the present method.

Thus, the present method is useful to treat subjects afflicted with or at risk of developing a gluten-induced disease with one or more AhR activating agents such as AhR agonists, bacterial probiotic combinations, IL-22 agonist, polypeptide or IL-22 nucleic acid, to increase expression of AhR pathway genes, lower gut dysfunction and reduced markers of acute inflammation in the GI tract.

In another aspect of the invention, a method of monitoring the treatment of a gluten-related disease, such as CeD, in a subject is provided. The method comprises: i) determining the AhR activity of the microbiota in a first biological sample obtained from the subject by performing the method of the invention; ii) administering to the subject at least one agent that binds to the aryl hydrocarbon receptor (AhR) to result in dissociation of the AhR from chaperones to permit dimerization of the AhR with AhR nuclear translocator (ARNT), for example, an AhR agonist, a bacterial probiotic, an IL-17 antagonist and an IL-22 polypeptide; iii) determining the AhRf activity of the microbiota in a second biological sample obtained from the subject; and iv) comparing the results determined in step i) with the results determined in step iii), wherein a difference between said results is indicative of the effectiveness of the treatment. Specifically, an increase in the AhR activity from the first sample to the second sample of the subject indicates that the treatment is effective.

I. Definitions

Unless otherwise indicated, the definitions and embodiments described in this and other sections are intended to be applicable to all embodiments and aspects of the present application herein described for which they are suitable as would be understood by a person skilled in the art.

As used herein, the “reference value” refers to a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the AhR activation levels (obtained according to the method of the invention) with a defined threshold value. In one embodiment of the present invention, the threshold value is derived from the AhR activation level (or ratio, or score) determined in a feces sample derived from one or more subjects having Celiac Disease (CeD) with abnormal microbiota exhibiting an impaired production of AhR ligands. Furthermore, retrospective measurement of the AhR activation level (or ratio, or scores) in properly banked historical subject samples may be used in establishing these threshold values.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise. For example, an embodiment including “an AhR agonist” should be understood to present certain aspects with one substance or two or more additional substances.

The term “and/or” as used herein means that the listed items are present, or used, individually or in combination. In effect, this term means that “at least one of” or “one or more” of the listed items is used or present.

### EXAMPLES

The following non-limiting examples are illustrative of the present application:

### Example 1. Trp Induces the AhR Pathway in NOD/DQ8 Mice Prior to and after Gluten-Sensitization

### Methods

Female and male 8-12 week old SPF NOD AB° DQ8 (NOD/DQ8) mice were maintained on a gluten-free diet (Envigo, TD.05620) and bred in a conventional SPF facility. All mice had unlimited access to food and water. NOD/DQ8 mice were fed a customized version of Envigo TD.01084 in which tryptophan concentration was adjusted at 0.1% (low Trp diet) or 1% (high Trp diet) (Table 1). Three weeks after the beginning of the diets, mice were either gluten sensitized and challenged or sacrificed and samples were collected to measure the effect of tryptophan supplementation on AhR activation.

TABLE 1
Low and High tryptophan (Trp) diet composition

| | Teklad diet number | |
|---|---|---|
| | Diet short name | |
| | TD.170282 | TD.170282 |
| | Low Trp | High Trp |
| | g/kg | g/kg |
| Sucrose | 344.5 | 348.68 |
| Corn starch | 150 | 150 |
| Maltodextrin | 150 | 150 |
| Soybean oil | 80 | 80 |
| Cellulose | 30 | 30 |
| Mineral mix, AIN-93M-MX (94049) | 35 | 35 |
| Calcium phosphate, monobasic, | 8.2 | 8.2 |
| monohydrate | | |
| Vitamin mix, AIN-93M-VX (94047) | 19.5 | 19.5 |
| Choline Bitartrate | 2.7 | 2.7 |
| TBHQ, antioxidant | 0.02 | 0.02 |
| Red food color | 0.1 | — |
| Yellow food color | — | 0.1 |
| L-Alanine | 3.5 | 3.5 |
| L-Arginine HCl | 12.1 | 12.1 |
| L-Asparagine | 6 | 6 |
| L-Aspartic Acid | 3.5 | 3.5 |
| L-Cysteine | 3.5 | 3.5 |
| L-Glutamic acid | 41.18 | 28 |
| Glycine | 23.3 | 23.3 |
| L-Histidine HCl, monohydrate | 4.5 | 4.5 |
| L-Isoleucine | 8.2 | 8.2 |
| L-Leucine | 11.1 | 11.1 |
| L-Lysine HCl | 18 | 18 |
| L-Methionine | 8.2 | 8.2 |
| L-Phenylalanine | 7.5 | 7.5 |
| L-Proline | 3.5 | 3.5 |
| L-Serine | 3.5 | 3.5 |
| L-Threonine | 8.2 | 8.2 |
| L-Tryptophan | 1 | 10 |
| L-Tyrosine | 5 | 5 |
| L-Valine | 8.2 | 8.2 |
| Protein (% by weight) | 15.4 | 15.3 |
| Protein (% kcal from) | 15.6 | 15.6 |
| Carbohydrate (% by weight) | 64.8 | 65.3 |
| Carbohydrate (% kcal from) | 66 | 66.2 |
| Fat (% by weight) | 8 | 8 |
| Fat (% kcal from) | 18.3 | 18.3 |
| Kcal/g | 3.9 | 3.9 |

*Diets are an isonitrogenous/isocaloric modification of TD.01084.

Mice were sensitized with 500 ug of sterilized pepsin-trypsin digest of gliadin (PT-gliadin) and 25 ug of cholera toxin by oral gavage once a week for 3 weeks, to break oral tolerance to gliadin. Following PT-gliadin sensitization, mice were challenged by oral gavage with 10 mg of sterile gluten dissolved in acetic acid three times a week for 3 weeks (“gluten treatment”). Mice were sacrificed 18 to 24 hours following the final challenge and samples were collected to measure the effect of tryptophan supplementation on AhR activation and gluten induced immunopathology.

The AhR activity of mouse stool samples was assessed through luciferase assay using a H1L1.1c2 cell line, containing a stably integrated dioxin-response element (DRE)-driven firefly luciferase reporter plasmid pGudLucl. These cells were seeded into a 96-well plate and treated with stool suspensions in DMEM and after 24 h of incubation luciferase activity was measured using a luminometer. The results were reported as fold changes based on the negative luciferase activity of the control medium (DMEM). All values were normalized based on the cytotoxicity of the samples using the Lactate Dehydrogenase Activity Assay.

The metabolite concentrations in the stool samples were determined by a specific method using HPLC-coupled to high resolution mass spectrometry. IDO activity was assessed by measurement of Kyn/Trp ratio.

Total DNA was extracted from mouse fecal samples, were sequenced and analyzed. Briefly, the V3 region of the 16S ribosomal RNA gene was amplified and sequenced on the Illumina MiSeq sequencing system. Sequences obtained were aligned to each other using the Paired-End Read (PEAR) merger. Clustering of reads into operational taxonomic units was performed using the Quantitative Insights into Microbial Ecology (QIIME) version 1.8.0 and data was loaded into R using the phyloseq package for downstream processing. A total of 4,012,804 reads were obtained with an average of 74,311.19 per sample ranging from 11512 to 169,626 per sample.

AhR activation in duodenal biopsies was measured through qPCR to assess the expression of AhR pathway genes: AhR, CYP1A1, IL-22 and IL-17. IL-15 gene expression was also measured by qPCR as an inflammation marker. Total RNA was isolated from mouse duodenum samples using RNeasy Mini Kit (Qiagen, Hilden, Germany) with the DNase treatment, according to the manufacturer's instructions. RNA concentration was determined using a NanoDrop spectrophotometer (Qiagen). Quantitative RT-PCR was performed using iScript Reverse Transcriptase (Bio-Rad) and then a SsoFast EvaGreen Supermix (Bio-Rad) in a Mastercycler ep realplex apparatus (Eppendorf) with specific mouse oligonucleotides. Additional information on the primers used is available in Table 2. qPCR data were analyzed using the 2-ΔΔCt quantification method with mouse Gapdh as the endogenous control.

TABLE 2
qPCR primers used for gene expression analysis in mice.
Mouse

| | | |
|---|---|---|
| GAPDH | FW | 5′-AACTTTGGCATTGTGGAAGG-3′ (SEQ ID NO. 1) |
| | RV | 5′-ACACATTGGGGGTAGGAACA-3′ (SEQ ID NO. 2) |
| AhR | FW | 5′-GAGCTTCTTTGATGGCGCTG-3′ (SEQ ID NO. 3) |
| | RV | 5′-GTCCACTCCTTGTGCAGAGT-3′ (SEQ ID NO. 4) |
| IL-17A | FW | 5′-TTTAACTCCCTTGGCGCAAAA-3′ (SEQ ID NO. 5) |
| | RV | 5′-CTTTCCCTCCGCATTGACAC-3′ (SEQ ID NO. 6) |
| IL-22 | FW | 5′-CATGCAGGAGGTGGTACCTT-3′ (SEQ ID NO. 7) |
| | RV | 5′-CAGACGCAAGCATTTCTCAG-3′ (SEQ ID NO. 8) |
| IL-15 | FW | 5′-CATTTTGGGCTGTGTCAGTG-3′ (SEQ ID NO. 9) |
| | RV | 5′-GCAATTCCAGGAGAAAGCAG-3′ (SEQ ID NO. 10) |
| Cyp1A1 | FW | 5′-ACATTGTGCCTGCCTCCTAC-3′ (SEQ ID NO. 11) |
| | RV | 5′-GTAGGGTGAACAGAGGTGCC-3′ (SEQ ID NO. 12) |
| AhRR | FW | 5′-CCATTCAGAAGCGCCTTGCAG-3′ (SEQ ID NO. 13) |
| | RV | 5′-AGGCAGCGAACACGACAAAT-3′ (SEQ ID NO. 14) |
| IL-6 | FW | 5′-TGTGCAATGGCAATTCTGAT-3′ (SEQ ID NO. 15) |
| | RV | 5′-CCAGAGGAAATTTTCAATAGGC-3′ (SEQ ID NO. 16) |

*FW = Forward sequence; RV = Reverse sequence.

Gut barrier function was assessed by a Ussing chamber technique. Briefly, sections of jejunum from each mouse were mounted in a Ussing Chamber with an opening of 0.6 cm². Net active transport across the epithelium was measured via a short circuit current response (Isc, μA) injected through the tissue under voltage-clamp conditions. Baseline Isc (μA/cm²) was recorded at equilibrium 20 min after mounting jejunum sections. Mucosal-to-serosal transport of macromolecules was assessed by adding ⁵¹Cr-EDTA in the luminal side of the chamber. ⁵¹Cr-EDTA fluxes were calculated by measuring the proportion of radioactive ⁵¹Cr-EDTA detected in the serosal side of the chamber after 2 hours compared to the radioactive ⁵¹Cr-EDTA placed in the luminal side at the beginning of experiment.

For histology and immunohistochemistry studies, cross-sections of the proximal small intestine were fixed in 10% formalin and embedded in paraffin. Enteropathy was determined by measuring villus-to-crypt (V/C) ratios in a blinded fashion. The presence of IELs (intraepithelial lymphocytes) in the sections was determined by immunostaining for CD3+ cells. Briefly, sections were stained with polyclonal rabbit anti-mouse CD3 (Dako) and in each section, and intraepithelial lymphocytosis was determined by counting CD3+ IELs per 20 enterocytes in five randomly chosen villus tips.

GrapPad Prism version 6.0 was used for all analyses and preparation of graphs. The data are presented as median with interquartile range and whiskers extending from minimum to maximum or mean±SEM. Normal distribution was determined by D'Agustino-Pearson omnibus normality test, Shapiro-Wilk test and Kolmogorov-Smirnov test with Dallal-Wilkinson-Lillie correction. For data sets that failed normality tests, nonparametric tests were used to analyze significant differences. Multiple comparisons were evaluated statistically by one-way ANOVA and post hoc Tukey test or nonparametric Kruskal-Wallis test followed by a post hoc Dunn's test. For comparisons between two groups, significance was determined using the two-tailed Student's t-test or nonparametric Mann-Whitney test. Statistical outliers (using the ROUT method) where technical issues were encountered, such as poor RNA quality, poor tissue quality for Ussing chambers, or poor histological orientation were removed from analysis. Exact numbers are provided in each figure legend. Differences corresponding to P<0.05 were considered significant.

For microbiota analysis, R Statistics, with the stats and vegan packages, was used to perform the statistical analysis. Data transformation was used when required and possible to achieve a normal distribution (logarithmic, square root, inversion, and inverted logarithm). Differences between whole bacterial communities were tested by permutational multivariate analysis of variance (PERMANOVA) calculated using an unweighted UniFrac distance. Multiple comparisons were evaluated statistically by one-way ANOVA or Kruskall-Wallis. Statistically significant differences were then evaluated by two-tailed Student's t-test or Wilcoxon rank-sum test and multiple testing was corrected via false discovery rate (FDR) estimation.

### Results

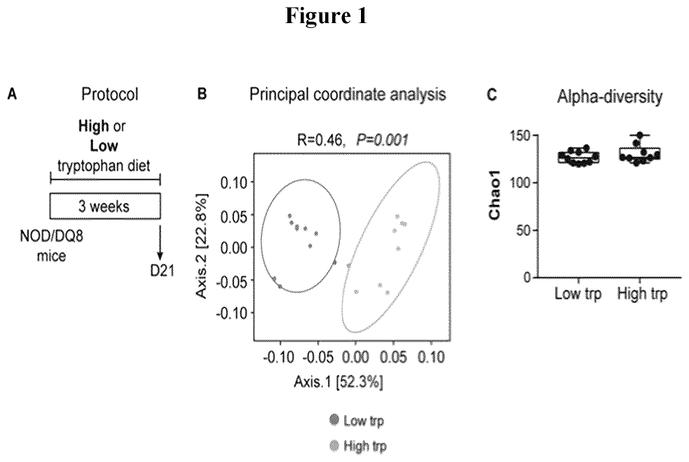

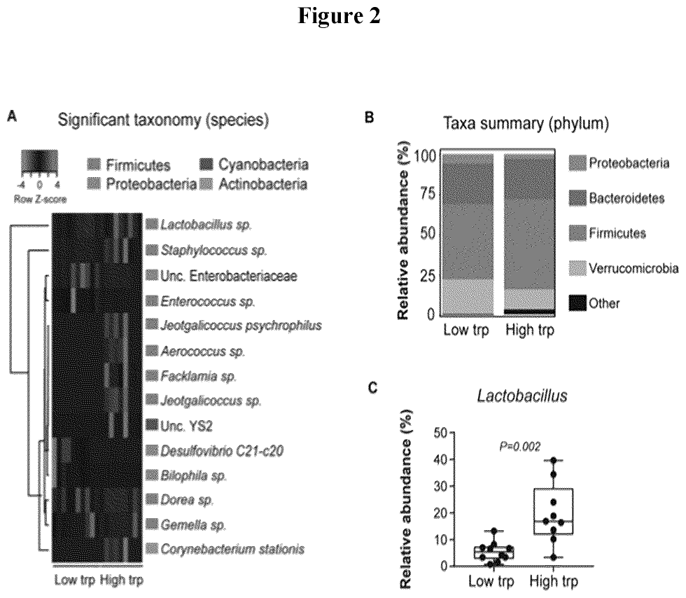

The capacity of an enriched Trp diet to shape the microbiota after 3 weeks of dietary intervention in NOD/DQ8 mice before gluten treatment was first investigated. Mice were fed a diet containing either low or enriched Trp concentration (FIG. 1A) for 3 weeks and the fecal microbiota composition was analyzed. The principal coordinate analysis revealed a difference in microbiota profiles between mice fed low and enriched Trp diet (FIG. 1B) but no difference in alpha diversity was observed (FIG. 1C). The microbiota of mice fed the enriched Trp diet showed a lower relative abundance of bacteria belonging to the Proteobacteria phylum such as Bilophila, Desulfovibrio and Enterobacteriaceae, and a higher abundance of bacteria belonging to the Firmicutes phylum such as Lactobacillus, Aerococcus, Facklamia, Jeotgalicoccus and Staphylococcus (FIG. 2). These data suggest that high Trp diet favors the growth of bacteria considered beneficial, such as Lactobacillus, able to metabolize Trp into AhR ligands, at the expense of potentially pro-inflammatory bacteria belonging to Proteobacteria.

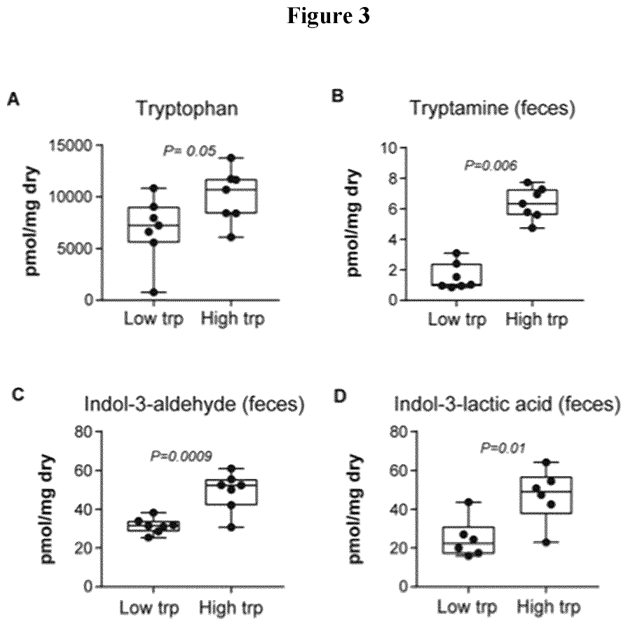

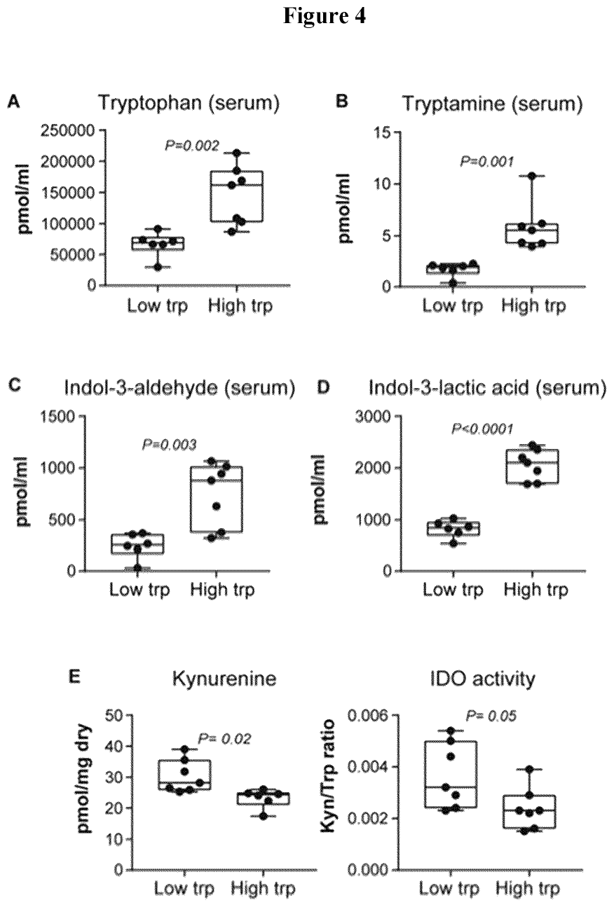

In measuring Trp metabolite concentrations, it was found that Trp level was higher in feces of mice fed the enriched Trp diet (FIG. 3A). This was associated with increased concentrations of AhR agonists including, tryptamine, indole-3-aldehyde and indole-3-lactic acid (FIG. 3B-D). A higher concentration of Trp and AhR agonists was also observed in the serum of mice fed the enriched Trp diet (FIG. 4). In contrast, the concentration of kynurenine (Kyn), a Trp metabolite produced mainly by IDO 1 enzyme in host cells and implicated in chronic inflammation, was higher in feces of mice fed the low Trp diet (FIG. 4E). Finally, the IDO activity determined by Kyn/Trp ratio, was higher in mice fed the low Trp diet (FIG. 4E). Thus, the metabolomic profile indicates that an enriched Trp diet shapes the gut microbiota to produce AhR ligands and potentially decreases intestinal IDO 1 activity.

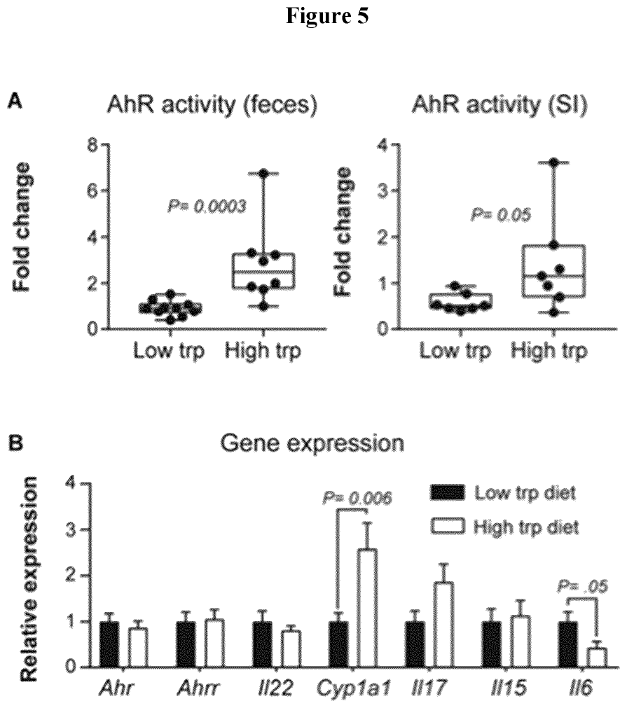

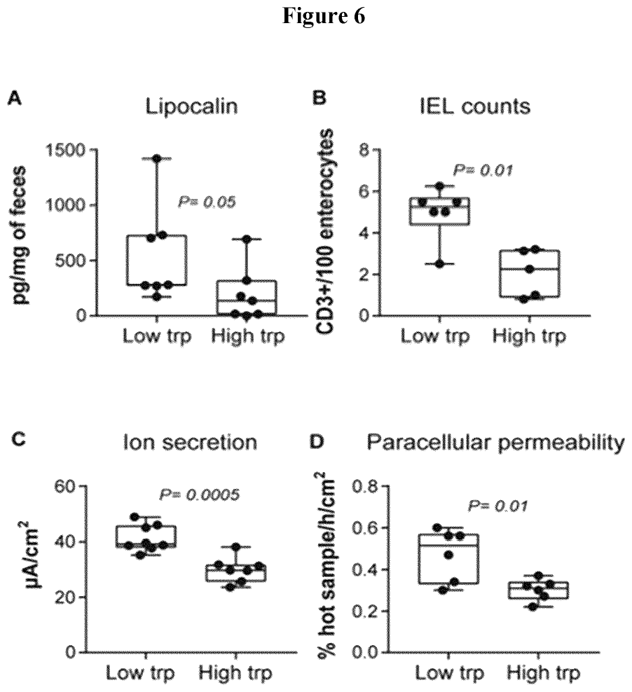

To investigate the functional relevance of these findings, the capacity of the microbiota to activate AhR was determined. Fecal and small intestinal contents from mice fed the enriched Trp diet induced greater activation of AhR than contents from mice fed the low Trp diet (FIG. 5A). An upregulation of Cyp1a1, an AhR target gene, in the duodenum of mice fed the enriched Trp diet was also detected (FIG. 5B). Lower levels of fecal lipocalin-2 (Lcn2), duodenal 116 expression and IEL counts, as well as decreased ion transport and paracellular permeability was observed in mice fed the enriched Trp diet, suggesting an anti-inflammatory effect of the diet (FIG. 6). Thus, the production of AhR agonists by the gut microbiota of high Trp diet-fed mice, induces AhR activation which may promote intestinal homeostasis.

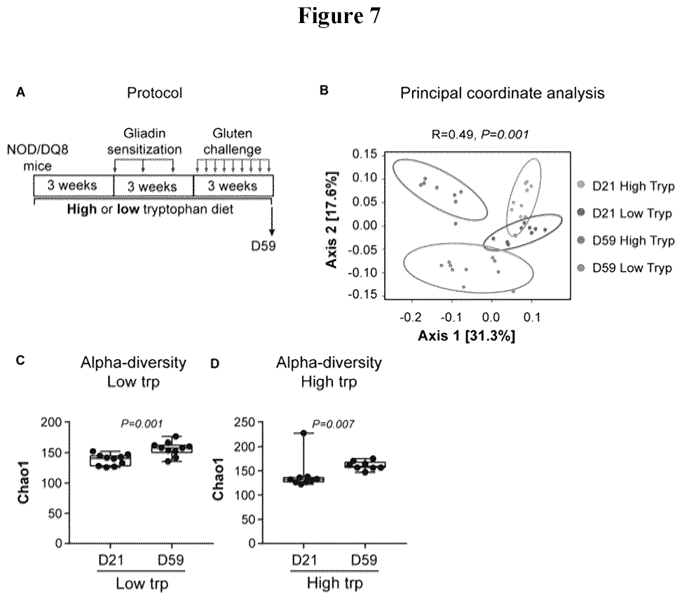

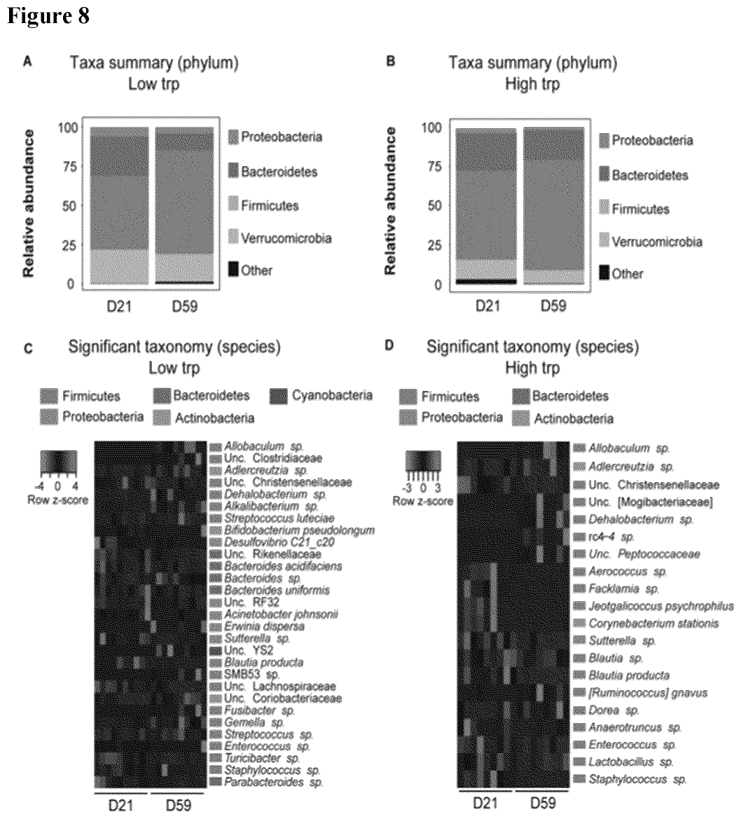

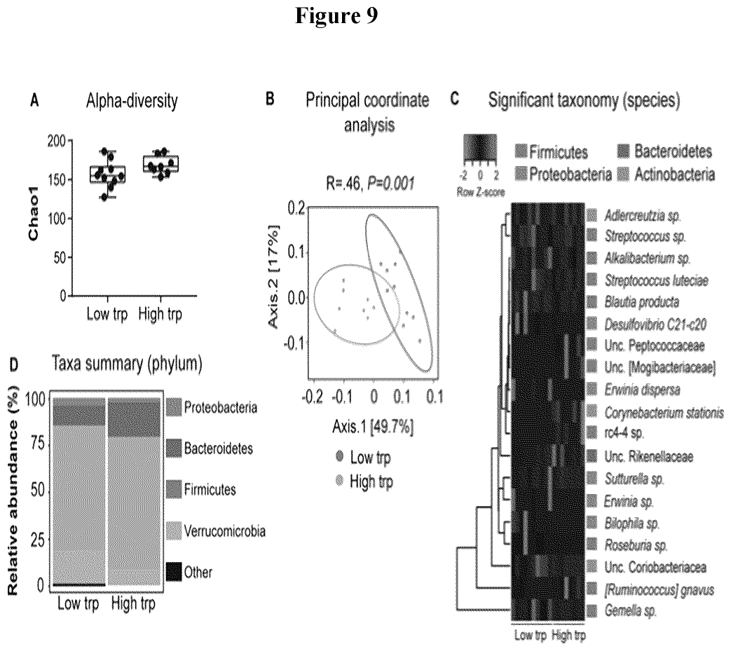

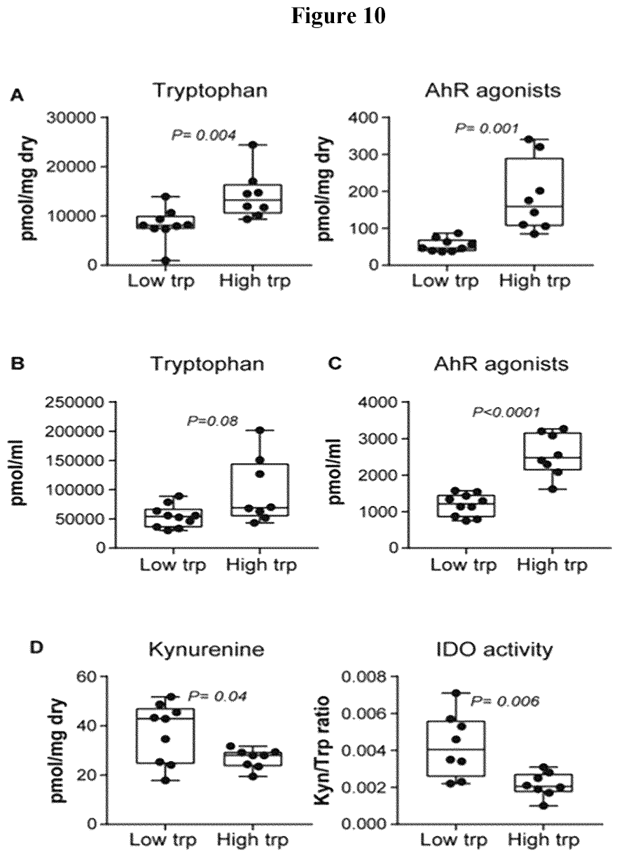

Gluten sensitization and challenge (D59; “gluten-treatment”; FIG. 7A), regardless of dietary intervention, shifted fecal microbiota composition (FIG. 7B) and alpha diversity (FIGS. 7B and 7C) leading to increased abundance of bacteria from the Firmicutes phylum and lower abundance of bacteria belonging to the Bacteroidetes phylum (FIG. 8). Compared with the low Trp diet, enriched Trp did not impact alpha diversity (FIG. 9A) but principal coordinate analysis revealed significantly different clustering between the two groups (FIG. 9B). Differential analysis confirmed this effect with a relative increase in bacteria belonging to the Bacteroidetes and Firmicutes phyla at the expense of bacteria from the Proteobacteria and Verrucomicrobia phyla (FIGS. 9C and 9D). At a species level, abundance of Bilophila, Desulfovibrio, Suturella and Erwinia, from the Proteobacteria phylum, was lower in the mice fed the enriched Trp diet (FIG. 9C). Among Firmicutes, Ruminococcus gnavus (FIG. 9C), known to produce AhR ligands, was increased in mice treated with the enriched Trp diet. Accordingly, levels of Trp and AhR agonists in feces and serum were higher in enriched Trp diet-fed mice (FIG. 10A to 10C). A decrease in kynurenine concentration and IDO activity in feces of enriched Trp diet-fed mice compared with low Trp diet-fed mice was also observed (FIG. 10D).

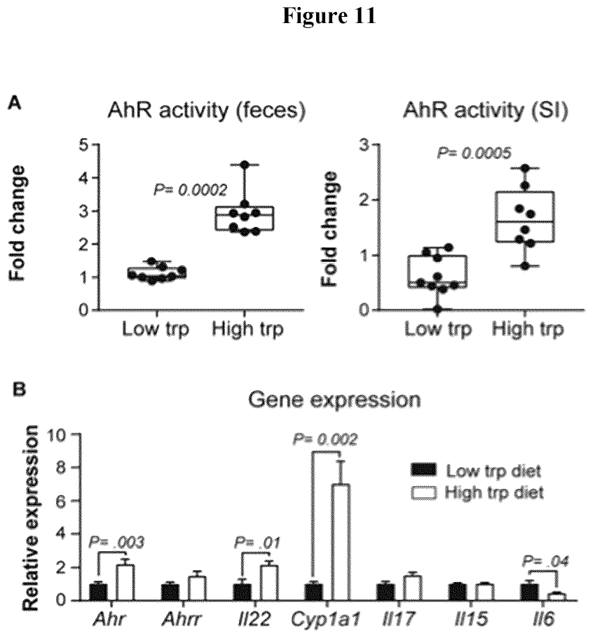

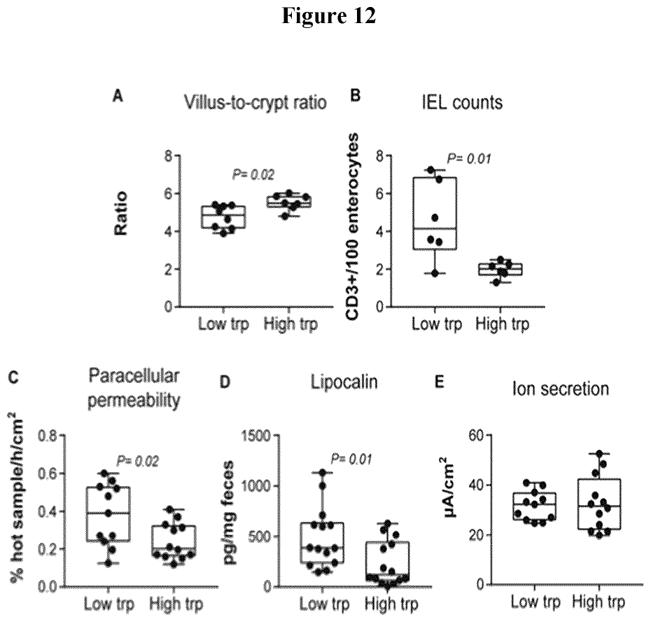

Taken together these results suggest that gluten exposure was a major driver of microbiota shift, independently of Trp content in diet; however, the enriched Trp diet maintained the functional capacity to produce AhR ligands and promoted a microbiota structural profile associated with beneficial bacteria capable of AhR ligand production. The capacity of small intestine and colon content to activate AhR was higher in enriched Trp diet-fed mice compared with low Trp diet-fed mice, (FIG. 11A) and this was paralleled by higher duodenal expression of AhR, and AhR target genes such as Cyp1a1 and I122 (FIG. 11B). The enriched Trp diet also improved villus-to-crypt (V/C) ratios, lowered IEL counts, intestinal paracellular permeability, fecal Lcn2 levels and duodenal I16 expression (FIG. 12). Collectively, this suggests that both impaired Trp metabolism by the gut microbiota and higher Trp metabolism by host cells can lead to defective AhR signaling and increased production of kynurenine metabolites that are associated with an inflammatory state. These results support that an enriched Trp diet improves gluten immunopathology in NOD/DQ8 mice by inducing and maintaining an intestinal microbiota able to produce AhR agonists.

A diet enriched in Trp, using a mouse model that develops moderate inflammation upon gluten sensitization and challenge, shifts the gut microbiota towards a higher abundance of Lactobacillus and Ruminococcus gnavus, which are known AhR ligand producers. Metabolomic and transcriptomic analysis indicates that this shift is accompanied by an increased production of AhR ligands, as well as an activation of the AhR pathway, which promotes homeostasis and ameliorates gluten immunopathology in mice expressing CeD risk genes.

Thus, it is herein demonstrated that AhR agonists produced from tryptophan metabolism can rescue AhR activation and reduce gluten-induced immunopathology.

### Example 2. AhR Activity and Trp Metabolism are Disrupted in CeD Patients

Methods

Eleven patients (7 females, mean age of 36.8 years) with a positive TG2 test were recruited after confirming active CeD by the presence of duodenal atrophy (>Marsh Ma). Patients with concurrent or past history of inflammatory bowel disease (Crohn's disease, ulcerative or undetermined colitis), as determined by laboratory, endoscopic or bowel imaging, were excluded. Sixteen other patients (9 females, mean age of 51 years) undergoing endoscopy, in which organic disease including peptic ulcer, reflux disease, inflammatory bowel disease and celiac disease were ruled out, were recruited as non-celiac controls. Characteristics of the subjects and clinical cohort are shown in Table 3. Six patients (3 with active CeD and 3 non-celiac controls) were taking a proton pump inhibitor in the month before enrolment. Patients taking immunosuppressants, glucocorticosteroids, antibiotics or probiotics were excluded. When possible, genetic analysis was performed for celiac risk genes (HLA-DQA1 and HLA-DQB1 genes). All the participants signed written informed consent; fecal samples were collected and kept frozen and stored until analysis.

TABLE 3
Characteristics of the subjects and clinical cohort.

| Diag- | | | | | Marsh | Bristol |
|---|---|---|---|---|---|---|
| nosis | HLA | Age | Sex | TG2 | scale | Scale |
| CeD | Not determined | 33 | M | Positive | 3c | 6 |
| CeD | Not determined | 26 | F | Positive | 3a | 5 |
| CeD | DQ2 homozygous | 46 | M | Positive | 3a | 5 |
| CeD | Not determined | 34 | F | Positive | 3a | 6 |
| CeD | DR3-DQ2 | 55 | M | Positive | 3c | 4 |
| | heterozygous | | | | | |
| CeD | DR3-DQ2 | 37 | F | Positive | 3a | 7 |
| | heterozygous/ | | | | | |
| | DR4-DQ8 | | | | | |
| | heterozygous | | | | | |
| CeD | DR3-DQ2 | 30 | F | Positive | 3a | 4 |
| CeD | DQ2 homozygous | 31 | F | Positive | 3b | 5 |
| CeD | DR3-DQ2 | 52 | M | Positive | 3a | 5 |
| CeD | DQ2 homozygous | 29 | F | Positive | 3a | 6, 7 |
| CeD | DQ2 homozygous | 32 | F | Positive | 3a | 2, 3, 4 |
| Control | Not determined | 45 | F | Negative | Normal | 6 |
| Control | Not determined | 50 | M | Negative | Normal | 4 |
| Control | Not determined | 53 | M | Negative | Normal | 2 |
| Control | Not determined | 39 | F | Negative | Normal | 6 |
| Control | Not determined | 42 | M | Negative | Normal | 6 |
| Control | DR3-DQ2 | 72 | F | Negative | Normal | 4 |
| | heterozygous | | | | | |
| Control | DR4-DQ8 | 72 | F | Negative | Normal | 6 |
| | heterozygous | | | | | |
| Control | Not determined | 63 | F | Negative | Normal | 4 |
| Control | Negative | 60 | M | Negative | Normal | 2 |
| Control | Not determined | 51 | F | Negative | Normal | 6 |
| Control | Not determined | 63 | F | Negative | Normal | 2 |
| Control | Not determined | 63 | M | Negative | Normal | 2 |
| Control | Not determined | 45 | M | Negative | Normal | 1 |
| Control | Not determined | 18 | F | Negative | Normal | 4 |
| Control | Negative | 45 | F | Negative | Normal | 6 |
| Control | DR7-DQ2 | 36 | M | Negative | Normal | 6 |
| | heterozygous | | | | | |

*CeD = Celiac Disease

The AhR activity of fresh human stool samples was assessed using the luciferase assay and a H1L1.1c2 cell line containing a stably integrated dioxin-response element (DRE)-driven firefly luciferase reporter plasmid pGudLucl. These cells were seeded into a 96-well plate and treated with stool suspensions in DMEM and after 24 h of incubation, luciferase activity was measured using a luminometer. The results were reported as fold changes based on the negative luciferase activity of the control medium (DMEM). All values were normalized based on the cytotoxicity of the samples using the Lactate Dehydrogenase Activity Assay.

The metabolite concentrations in the stool samples were determined by a specific method using HPLC-coupled to high resolution mass spectrometry. IDO activity was assessed by measurement of Kyn/Trp ratio. AhR activation in duodenal biopsies was measured through qPCR to assess the expression of AhR pathway genes: AhR, CYP1A1, IL-22 and IL-17. IL-15 gene expression was also measured by qPCR as inflammation marker. Total RNA was isolated from human duodenum samples using the RNeasy Mini Kit (Qiagen, Hilden, Germany) with DNase treatment, according to the manufacturer's instructions. RNA concentration was determined using a NanoDrop spectrophotometer. Quantitative RT-PCR was performed using iScript Reverse Transcriptase and then a SsoFast EvaGreen Supermix in a Mastercycler ep realplex apparatus with specific human oligonucleotides. Additional information on the primers used is available in Table 4. qPCR data were analyzed using the 2^{-ΔΔCt} quantification method with human Gapdh as the endogenous control.

TABLE 4
qPCR primers used for human gene expression analysis.
Human

| | | |
|---|---|---|
| GAPDH | FW | 5′-CGGAGTCAACGGATTTGGTCGTAT-3′ (SEQ ID NO. 17) |
| | RV | 5′-AGCCTTCTCCATGGTGGTGAAGAC-3′ (SEQ ID NO. 18) |
| AhR | FW | 5′-CCACTTCAGCCACCATCCAT-3′ (SEQ ID NO. 19) |
| | RV | 5′-AAGCAGGCGTGCATTAGACT-3′ (SEQ ID NO. 20) |
| IL-17A | FW | 5′-AACCGATCCACCTCACCTTG-3′ (SEQ ID NO. 21) |
| | RV | 5′-TCTCTTGCTGGATGGGGACA-3′ (SEQ ID NO. 22) |
| IL-22 | FW | 5′-CGTTCGTCTCATTGGGGAGA-3′ (SEQ ID NO. 23) |
| | RV | 5′-ACATGTGCTTAGCCTGTTGC-3′ (SEQ ID NO. 24) |
| IL-15 | FW | 5′-GGCCCAAAGCACCTAACCTAT-3′ (SEQ ID NO. 25) |
| | RV | 5′-TGCATCTCCGGACTCAAGTG-3′ (SEQ ID NO. 26) |
| CYP1A1 | FW | 5′-CTACCCAACCCTTCCCTGAAT-3′ (SEQ ID NO. 27) |
| | RV | 5′-CGCCCCTTGGGGATGTAAAA-3′ (SEQ ID NO. 28) |
| AhRR | FW | 5′-GGCTGCTGTTGGAGTCTCTT-3′ (SEQ ID NO. 29) |
| | RV | 5′-CATCGTCATGAGTGGCTCGG-3′ (SEQ ID NO. 30) |

*FW = Forward sequence; RV = Reverse sequence.

### Results

To assess the relevance of gut microbiota-derived AhR ligands in CeD pathogenesis and the impact of impaired intestinal AhR activity and signaling, the concentrations of specific microbiota metabolites were examined in fecal samples and the AhR pathway gene expression was examined in duodenal biopsies from the human gastrointestinal (GI) tract of CeD patients and non-celiac controls. Fecal samples from CeD patients had a decreased ability to activate the AhR pathway (using the H1L1.1 c2 reporter cell line) while the intestinal biopsies showed significantly less expression of genes regulated by the AhR pathway based on qPCR.

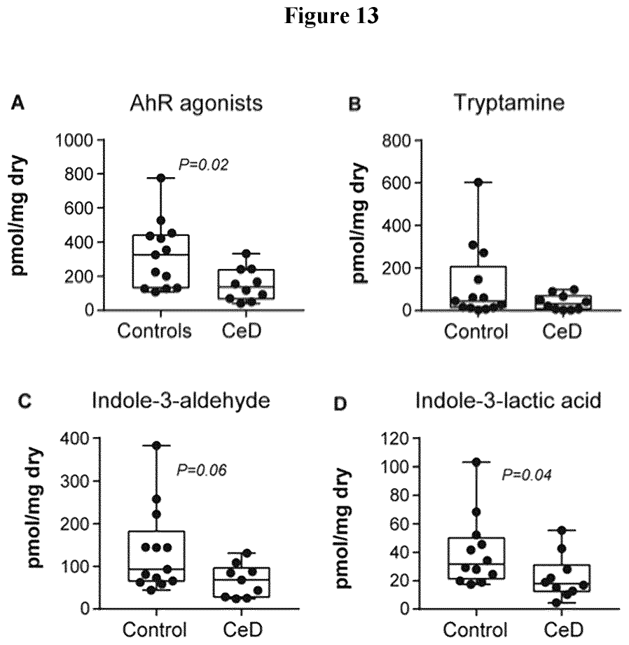

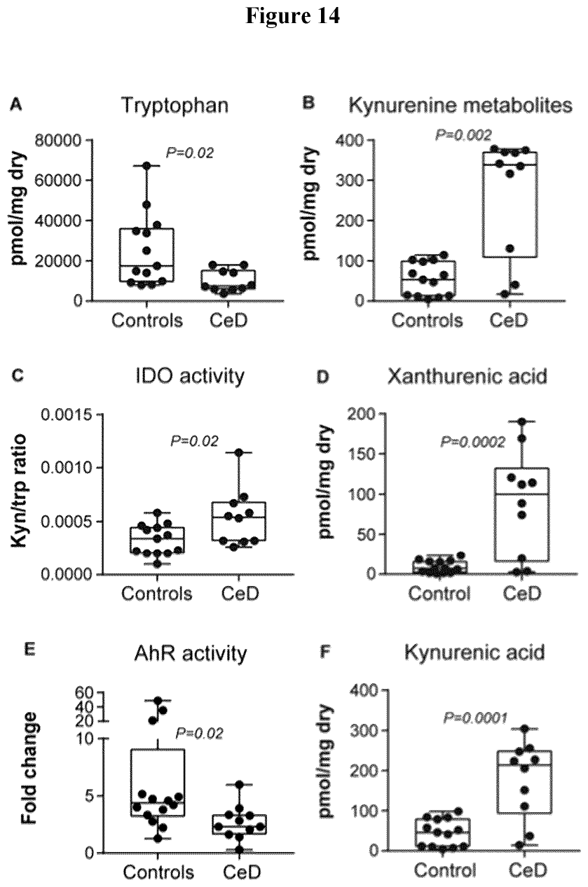

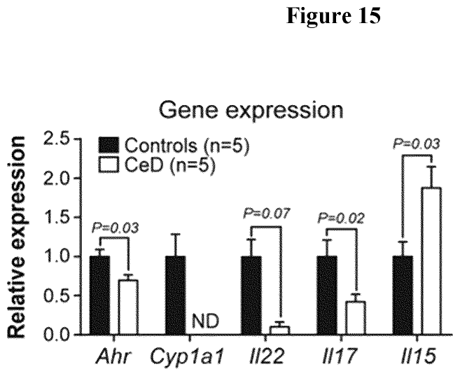

Metabolism of tryptophan by gut microbiota and the host cell produce metabolites that are known to be inducers (agonists) of the AhR pathway. Fecal samples from patients with CeD had lower concentrations of AhR agonists including tryptamine, indole-3-aldehyde and indole-3-lactic acid (FIG. 13). A higher concentration of Trp was observed in fecal samples from non-celiac controls compared with patients with CeD (FIG. 14A). In contrast, xanthurenic acid and kynurenic acid, products of Trp metabolism through the kynurenine pathway, and the IDO activity, were higher in fecal samples of CeD patients compared with healthy controls (FIG. 14B to 14F). Consistent with the impaired production of AhR ligands observed, fecal samples from patients with CeD had a lower capacity to activate AhR (FIG. 14F). Expression of AhR and AhR pathway genes such as Cyp1a1, I122 and I117 were decreased while the key cytokine in CeD, I115, was increased (FIG. 15).

Taken together, these clinical data suggest an impaired Trp metabolism by the gut microbiota in CeD patients, leading to defective AhR activation, but a higher Trp metabolism by host immune cells leading to the production of kynurenine metabolites, and likely related to an inflammatory state. Collectively, these results show that AhR activation and signaling are impaired in the GI tract of CeD patients. Decreased production of AhR agonists by the microbiota of CeD patients was detected and associated with an impaired AhR pathway to contribute to the gluten-induced immunopathology.

### Example 3. AhR Agonists Reduce Tut Dysfunction/Inflammation Due to Gluten Sensitization

Methods

Female and male 8-12 week old SPF NOD AB° DQ8 (NOD-DQ8) mice were maintained on a gluten-free diet (Envigo, TD.05620) and bred in a conventional SPF facility. All mice had unlimited access to food and water. NOD-DQ8 mice were sensitized for 3 weeks with 500 mg of sterilized pepsin-trypsin digest of gliadin (PT-gliadin) and 25 mg of cholera toxin by oral gavage once a week for 3 weeks, to break oral tolerance to gliadin. After this period, mice were challenged by oral gavage with 10 mg of sterile gluten dissolved in acetic acid three times a week for 3 weeks (“gluten treatment”). During the experiment one group was injected i.p. with an AhR agonist, 6-formylindolo(3,2-b) carbazole (Ficz, 1 μg/mouse), diluted in DMSO while the control group received vehicle (DMSO) only at day 1, 10, 20 and 30. Mice were sacrificed 18 to 24 hours following the final gluten challenge.

### Results

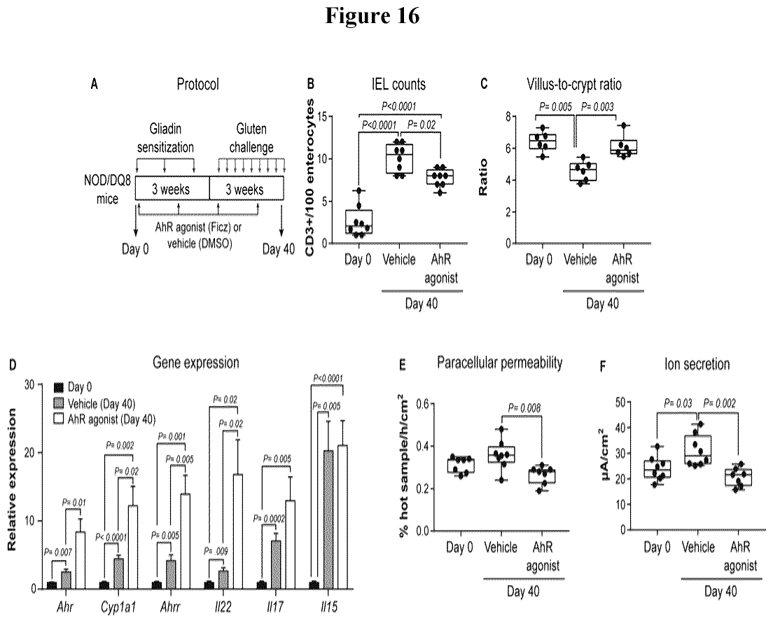

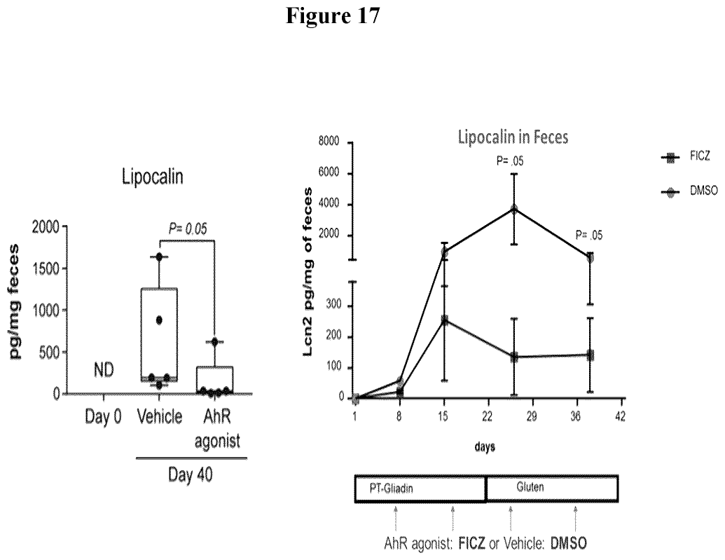

The therapeutic value of intestinal AhR activation, using an AhR agonist, the 6-formylindolo (3,2-b) carbazole (Ficz) was investigated (FIG. 16A). After gluten treatment, mice developed higher IEL counts, lower V/C ratios, increased Il15 expression, and barrier dysfunction (FIG. 16B to 16F and FIG. 17). Compared to basal conditions (day 0), gluten treated mice also developed an increased intestinal expression of AhR and AhR target genes such as Cyp1a1, Il22 and Il17, suggesting an overall state of immune activation (FIG. 16D). However, an even higher expression of these AhR target genes was observed in the group treated with Ficz, confirming the activation of AhR by the agonist (FIG. 16D). Ficz treatment did not affect intestinal IL-15 expression (FIG. 16D), but it improved IEL counts, V/C ratios, paracellular permeability, ion transport, and other markers of overall gut inflammation (FIG. 16B to 16F and FIG. 17).

Taken together, these results demonstrate that pharmacological modulation of the AhR pathway ameliorates gluten immunopathology in NOD/DQ8 mice. Thus, it is herein demonstrated that an AhR agonist can rescue AhR activation and IL-22 production, and reduce gluten-induced immunopathology.

### Example 4. Inoculation with Lactobacillus that Produce AhR Agonists Activates the AhR Pathway in a CeD Animal Model

### Methods

Female and male 8-12 weeks old SPF NOD AB° DQ8 (NOD/DQ8) mice were maintained on a gluten-free diet (Envigo, TD.05620) and bred in a conventional SPF facility. All mice had unlimited access to food and water. NOD/DQ8 mice were fed a customized version of Envigo TD.01084 in which tryptophan concentration was adjusted at 0.1% (low Trp diet) or 1% (high Trp diet) (Table 1). Three weeks after the beginning of the diets, mice were either gluten sensitized and challenged or sacrificed. During gluten challenge, mice of each diet group were gavaged 6 times a week for 3 weeks with 10⁹ colony-forming units of a combination of Lactobacillus reuteri CNCM-I5022 and L. reuteri CNCM-I5429. Bacteria were grown in MRS broth supplemented with cysteine (0.5 mg/mL) and Tween 80 (1 mg/mL) for 18-20 h. Oral gavage with MRS was performed in control mice. Mice were sacrificed 24 hours following the final challenge and samples were collected to measure the effect of tryptophan supplementation+lactobacillus on AhR activation and gluten-induced immunopathology.

### Results

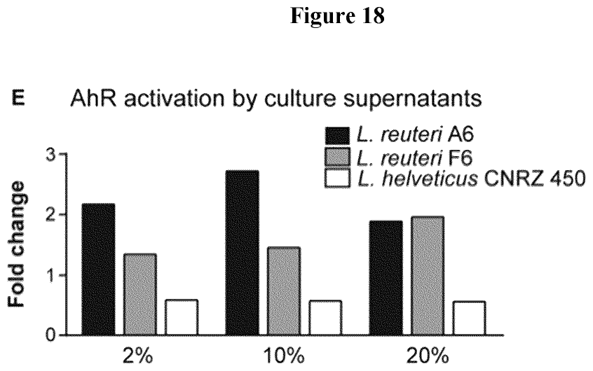

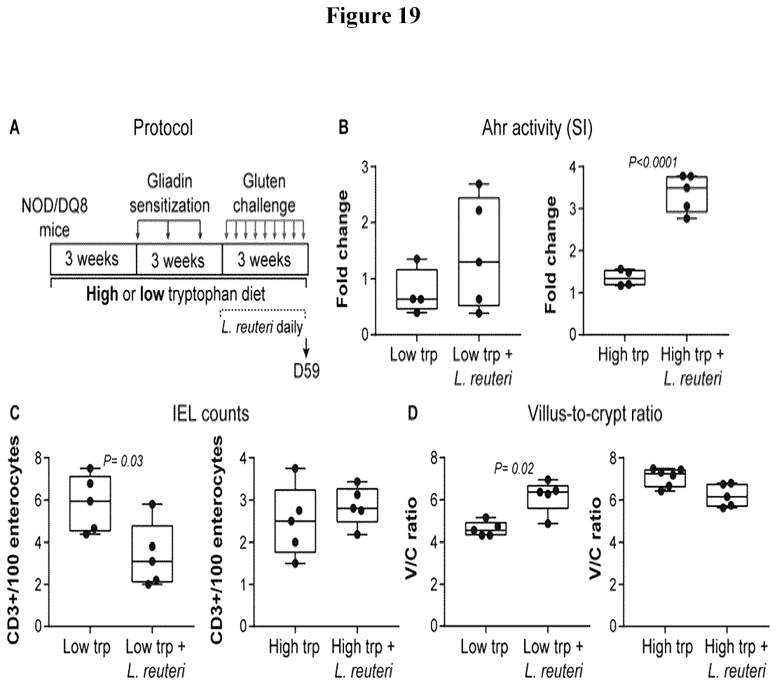

To investigate microbial modulation of AhR activity in the model, two previously isolated Lactobacillus reuteri strains that naturally exhibit high AhR-ligand production (FIG. 18) were administered to gluten treated NOD/DQ8 mice fed either a low or enriched Trp diet (FIG. 19A). L. reuteri supplementation increased the capacity of the small intestinal microbiota to activate AhR in mice fed both diets, but the increase was only significant in mice fed the enriched Trp diet (FIG. 19B). L. reuteri supplementation decreased IEL counts in mice fed a low Trp diet compared with mice treated with media (FIG. 19C). L. reuteri also improved V/C ratios in mice on a low Trp diet (FIG. 19D).

Taken together, these results suggest that even in the context of a low Trp diet, supplementation with L. reuteri is sufficient to produce AhR ligands that modulate gluten immunopathology. It was found that L. reuteri supplementation increased the capacity of the small intestinal microbiota to activate AhR, particularly in mice fed the enriched Trp diet, indicating a synergism between the substrate and its metabolizer. Thus, it is herein demonstrated that modulation of the AhR pathway with probiotic AhR ligand producers, alone or in combination with dietary Trp, improves gluten immunopathology.

In summary, the inventors identified a novel mechanism related to the impaired production of AhR ligands by the intestinal microbiota in CeD. In mice, it was shown that gluten-induced pathology can be reversed through a combination of tryptophan in the diet and probiotics that produce AhR ligands. Therefore, in addition to providing key evidence of the importance of the microbiota composition and function in CeD pathogenesis, this study suggests that Trp catabolites derived from the metabolic activity of the intestinal microbiota may be used as biomarkers for dysbiosis. These include tryptophan supplementation in combination with next generation probiotic organisms, such as Lactobacillus strains (e.g. L reuteri strains) that produce AhR ligands from the dietary substrate. Currently, the only treatment for CeD is a strict, life-long adherence to a gluten-free diet (GFD). However, many suffer from persistent symptoms, despite following a GFD. In addition, this approach may also be used as a preventative strategy in at-risk populations.

While the present invention has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE APPLICATION

Furumatsu, K. et al. A role of the aryl hydrocarbon receptor in attenuation of colitis. Dig. Dis. Sci. 2011, 56:2532-2544.
Galipeau H J, Rulli N E, Jury J, Huang X, Araya R, Murray J A, David C S, Chirdo F G, McCoy K D, Verdu E F: Sensitization to gliadin induces moderate enteropathy and insulitis in nonobese diabetic-DQ8 mice. J Immunol 2011, 187:4338e4346
Gao, X. et al. Metabolite analysis of human fecal water by gas chromatography/mass spectrometry with ethyl chloroformate derivatization. Anal. Biochem. 2009, 393:163-75.
He, G., Zhao, B. & Denison, M. S. Identification of benzothiazole derivatives and polycyclic aromatic hydrocarbons as aryl hydrocarbon receptor agonists present in tire extracts. Environ. Toxicol. Chem. 2011, 30:1915-25.
Ji T, Xu C, Sun L, Yu M, Peng K, Qiu Y. et al. Aryl Hydrocarbon Receptor Activation Down-Regulates IL-7 and Reduces Inflammation in a Mouse Model of DSS-Induced Colitis. Dig Dis Sci. 2015, 60:1958-66.
Kim C Y, Quarsten H, Bergseng E, et al. Structural basis for HLA-DQ2-mediated presentation of gluten epitopes in celiac disease. Proc Natl Acad Sci USA 2004, 101:4175-4179.
Lamas B, Natividad J M, Sokol 1H: Aryl hydrocarbon receptor and intestinal immunity. Mucosal Immunology 2018, 11:1024-38.
Lamas, B. et al. CARDS impacts colitis by altering gut microbiota metabolism of tryptophan into aryl hydrocarbon receptor ligands. Nat. Med. 2016, 22, 598-605.
Liu E, Lee H S, Aronsson C A, Hagopian W A, Koletzko S, Rewers M J, Eisenbarth G S, Bingley P J, Bonifacio E, Simell V et al.: Risk of pediatric celiac disease according to HLA haplotype and country. N Engl J Med 2014, 371:42-49
Ludvigsson J F, Leffler D A, Bai J C, et al. The Oslo definitions for coeliac disease and related terms. Gut 2013, 62:43-52.
Leonard M M, Vasagar B. US perspective on gluten-related diseases. Clin Exp Gastroenterol 2014, 7:25-37.
Safe S, Lee S O, Jin U H. Role of the aryl hydrocarbon receptor in carcinogenesis and potential as a drug target. Toxicol. Sci. 2013, 135:1-16.
Sollid L M: Molecular basis of celiac disease. Annu Rev Immunol 2000, 18:53-81
Verdu E F, Galipeau H J, Jabri B. Novel players in coeliac disease pathogenesis: role of the gut microbiota. Nat Rev Gastroenterol Hepatol 2015; 12:497-506

1. A method for treating a gluten-induced disease in a subject in need thereof comprising administering to the subject at least one agent that activates aryl hydrocarbon receptor.

2. The method of claim 1, wherein the agent binds to the AhR, causes dissociation of the AhR from chaperone molecules to permit dimerization of the AhR with AhR nuclear translocator (ARNT) to increase AhR activity.

3. The method of claim 1, wherein the agent that activates AhR is selected from the group consisting of an AhR agonist, a bacterial probiotic with AhR agonist activity, an interleukin-22 (IL-22) agonist, an IL-22 polypeptide, nucleic acid encoding an IL-22 polypeptide, an IL-17 antagonist and combinations thereof.

4. The method of claim 3, wherein said AhR agonist is selected from the group consisting of indole derivatives, tryptophan derivatives, tryptophan catabolites of the microbiota, flavonoids, biphenyls, polyphenolics, halogenated aromatic hydrocarbons, polycyclic aromatic hydrocarbons, polychlorinated dibenzodioxins, dibenzofurans, pyridines, benzimadazoles, methylenedioxybenzenes, AhR modulators (SAhRM) and natural AhR agonists (NAhRAs).

5. The method of claim 3, wherein the AhR agonist is selected from the group consisting of: indolocarbazole (ICZ), 6-formylindolo(3,2-b)carbazole (Ficz), tryptophan, kynurenine, kynurenic acid, indole-3-aldehyde (IAld), tryptamine, indole 3-acetate, 3-indoxyl sulfate, 2,3,7,8-tetrachlorodibenzo-p-dioxin (TCDD), quercetin, diosmin, tangeritin, tamarixetin, luteolin, myricetin, 3-methylcholanthrene, benzo[a]pyrene, benzanthracene, benzoflavone, caffeine and nicotine.

6. The method of claim 3, wherein the AhR agonist is selected from the group consisting of indole derivatives, tryptophan derivatives and tryptophan catabolites of the microbiota.

7. The method of claim 3, wherein the AhR agonist is selected from the group consisting of indolocarbazole (ICZ), 6-formylindolo(3,2-b)carbazole (Ficz), tryptophan, kynurenine, kynurenic acid, indole-3-aldehyde (IAld), tryptamine, indole 3-acetate, 3-indoxyl sulfate and 2,3,7,8-tetrachlorodibenzo-p-dioxin (TCDD).

8. The method of claim 3, wherein the AhR agonist is a selective AhR modulator (SAhRM) selected from diindolylmethane (DIM), carbidopa, methyl-substituted diindolylmethanes, dihalo- and dialkylDIM analogs, mexiletine, β-naphthoflavone (PNF), 5,6 benzoflavone (5,6 BZF), and 1,4-dihydroxy-2-naphthoic acid (DHNA).

9. The method of claim 1, wherein the agent that activates AhR is a bacterial probiotic or a prebiotic that stimulates an AhR-activating probiotic.

10. The method of claim 9, wherein said bacterial probiotic is selected from the group consisting of: Allobaculum, Lactobacillus reuteri, Lactobacillus taiwanensis, Lactobacillus johnsonii, Lactobacillus animalis, Lactobacillus murinus, Lactobacillus bulgaricus, Lactobacillus delbrueckii subsp. Bulgaricus, bacteria of the genus Adlercreutzia, bacteria of the phylum Actinobacteria, lactic acid bacterium, Streptococcus thermophilus, Bifidobacterium, Propionic acid bacterium, Bacteroides, Eubacterium, anaerobic Streptococcus, Enterococcus, Escherichia coli and combinations thereof.

11. The method of claim 9, wherein the bacterial probiotic comprises a Lactobacillus sp.

12. The method of claim 9, wherein said bacterial probiotic comprises bacteria selected from the group of bacterial probiotics deposited at the Collection Nationale de Cultures de Microorganismes (CNCM) having the CNCM deposit numbers: CNCM I-5019 (SB6WTD3, Lactobacillus taiwanensis), CNCM 1-5020 (SB6WTD4, Lactobacillus murinus), CNCM 1-5021 (SB6WTD5, Lactobacillus animalis), CNCM 1-5022 (SB6WTF6, Lactobacillus reuteri), and CNCM 1-5023 (SB6WTG6, Lactobacillus reuteri).

13. The method of claim 1, wherein the agent that activates AhR is a combination of an AhR agonist and a bacterial probiotic that activates AhR.

14. The method of claim 13, wherein the AhR agonist is an indole, tryptophan or a tryptophan catabolite.

15. The method of claim 13, wherein the bacterial probiotic comprises Lactobacillus.

16. The method of claim 1, wherein the gluten-induced disease is celiac disease (CeD), a T-cell—mediated enteropathy, dermatitis herpetiformis, gluten ataxia or a non-celiac gluten or wheat sensitivity.

17. The method of claim 1, wherein the gluten-induced disease is celiac disease.

18. A composition comprising an AhR agonist and a bacterial probiotic that activates AhR.

19. The composition of claim 18, wherein the AhR agonist is an indole, tryptophan or a tryptophan catabolite.

20. The composition of claim 19, wherein the AhR agonist is selected from the group consisting of indolocarbazole (ICZ), 6-formylindolo(3,2-b)carbazole (Ficz), tryptophan, kynurenine, kynurenic acid, indole-3-aldehyde (IAld), tryptamine, indole 3-acetate and 3-indoxyl sulfate.

21. The composition of claim 18, wherein the bacterial probiotic comprises Lactobacillus.

22. A method of preventing or treating gluten-induced disease in a subject in need thereof comprising the steps of: i) determining the Ahr activity of the microbiota in a biological sample obtained from the subject, ii) comparing the level determined at step i) with a predetermined reference value and iii) administering to the subject at least one AhR-activating agent selected from the group consisting of AhR agonists, bacterial probiotics with AhR agonist activity, and IL-22 agonist, polypeptide and/or IL-22-encoding nucleic acid when the level determined at step i) is lower than the predetermined reference value.

23. A method for monitoring the treatment of gluten-induced disease in a subject in need thereof, said method comprising the steps consisting of:
i) determining the AhR activity in a first biological sample obtained from the subject;
ii) administering to the subject at least one AhR-activating agent to the subject;
iii) determining the AhR activity second biological sample obtained from the subject; and
iv) comparing the results determined a step i) with the results determined at step iii) wherein a difference between said results is indicative of the effectiveness of the treatment.

24. A method of screening a candidate agent for use as a drug for the prevention or treatment of a gluten-induced disease comprising the steps of:
i) exposing a cell or tissue that expresses an AhR to a candidate agent under suitable conditions;
ii) measuring the AhR activity of the cell or tissue in the presence of the candidate agent; and
iii) identifying a candidate agent as a potential drug if the agent induces AhR activity.

25. Use of an agent that activates aryl hydrocarbon receptor (AhR) to treat a gluten-induced disease in a subject in need.

26. Use as defined in claim 25, wherein the agent that activates AhR is selected from the group consisting of an AhR agonist, a bacterial probiotic with AhR agonist activity, an interleukin-22 (IL-22) agonist, an IL-22 polypeptide, nucleic acid encoding an IL-22 polypeptide, an IL-17 antagonist and combinations thereof.

27. Use as defined in claim 26, wherein the agent comprises an AhR agonist and a bacterial probiotic that activates AhR.

28. Use as defined in claim 27, wherein the AhR agonist is an indole, tryptophan or a tryptophan catabolite, and the probiotic is a Lactobacillus sp.

29. Use as defined in claim 28, wherein the AhR agonist is selected from the group consisting of indolocarbazole (ICZ), 6-formylindolo(3,2-b)carbazole (Ficz), tryptophan, kynurenine, kynurenic acid, indole-3-aldehyde (IAld), tryptamine, indole 3-acetate and 3-indoxyl sulfate.

30. Use as defined in claim 25, wherein the gluten-induced disease is celiac disease (CeD), a T-cell˜mediated enteropathy, dermatitis herpetiformis, gluten ataxia or a non-celiac gluten or wheat sensitivity.
